(19) United States
(12) Patent Application Publication
(10) Pub. No.: US 2006/0106284 A1
(43) Pub. Date: May 18, 2006

(54) Communication method of storage device, image storage system and image storage device

(75) Inventors: Hideyuki Shouji, Tokyo (JP); Tomohiko Oda, Tokyo (JP); Yoshitaka Miyoshi, Tokyo (JP); Nobuyasu Ito, Tokyo (JP); Toshiro Ijichi, Tokyo (JP); Mutsumi Ohshima, Tokyo (JP); Tadao Eto, Tokyo (JP); Masashi Kikkawa, Tokyo (JP); Katsuyoshi Ishibashi, Tokyo (JP); Kazutaka Nakatsuchi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, TOKYO (JP)

(21) Appl. No.: 11/284,997

(22) Filed: Nov. 22, 2005

Related U.S. Application Data
(63) Continuation of application No. PCT/JP04/07395, filed on May 21, 2004.

(30) Foreign Application Priority Data
May 27, 2003 (JP) 2003-148802
May 27, 2003 (JP) 2003-148621
May 22, 2003 (JP) 2003-144668

(51) Int. Cl. A61B 1/00; A61B 1/04

(52) U.S. Cl. 600/118; 600/117; 600/101; 600/109

(57) ABSTRACT

This is the communication method of an image storage device for recording medical information. The communication method comprises a receiving information from a plurality of devices for providing at least one of setting information, status information, inspection image information and patient information, and transmits at least one of the setting information and status information or information obtained by processing the information, via a network, in addition to the inspection image information and patient information.

### CROSS REFERENCE TO RELATED APPLICATIONS

This application is a continuation application of PCT Application No. PCT/JP2004/007395 filed in Japan on May 21, 2004 and claims benefit of Japanese Applications No. 2003-144668 filed in Japan on May 22, 2003, No. 2003-148621 filed in Japan on May 27, 2003, and No. 2003-148802 filed in Japan on May 27, 2003, the entire contents of each of which are incorporated herein by their reference.

### BACKGROUND OF THE INVENTION

1. Field of the Invention

The present invention relates to a storage device for recording medical information, such as a medical image and the like.

2. Description of the Related Art

Conventionally, in medical institutions, such as a hospital and the like, a variety of medical devices are used, and an inspection, a diagnosis and a treatment are conducted. Each of these medical devices is connected to a peripheral device exclusive for the medical device (such as an image storage device, etc.), capable of obtaining information provided by the medical device, processing it, displaying it and so on, and is used as a system. The peripheral device is also one of them.

FIG. 1 shows one example of such a medical device and its peripheral device.

In FIG. 1, an electronic endoscope device 1, a supersonic wave endoscope device 2, a light source device 3 for an electronic endoscope device are examples of the medical device. An image storage device 1a and a display device 1b are example of the peripheral device exclusive for the electronic endoscope device 1, an image storage device 2a and a display device 2b are example of the peripheral device, exclusive for the supersonic wave endoscope device 2, and a light source device information acquisition device 3a for an electronic endoscope device and a display device 3b are examples of the peripheral device exclusive for the light source device 3 for an electronic endoscope device.

The electronic endoscope device 1, image storage device 1a and display device 1b are used for the system of the electronic endoscope device 1, and each device is configured by the specification unique to this system. In this system, an inspection image (inspection image information, which is an image obtained inspection), patient information peculiar to a patient, the setting information of the device 1, the status information of the device 1 or the like provided by the electronic endoscope device 1 is obtained by the image storage device 1a, and is displayed on the display device 1b as requested. Thus, for example, the inspection image and patient information of the device 1 can be read, its settings can be checked, and its status can be monitored.

The supersonic wave endoscope device 2, image storage device 2a and display device 2b are used for the system of the supersonic endoscope device 2, and each device is configured by the specification unique to this system. In this system, an inspection image, patient information provided by the supersonic endoscope device 2, the setting information of the device 2, the status information of the device 2 or the like is obtained by the image storage device 2a, and is displayed on the display device 2 as requested. Thus, for example, the inspection image and patient information of the device 2 can be read, its settings are checked, its status can be monitored and so on.

A system for obtaining and displaying an endoscopic image shot by an endoscope device like these systems is disclosed, for example, by Japanese Patent Application No. 7-141498 or the like.

The light source device 3 for an electronic endoscope device, light source device information acquisition device 3a for an electronic endoscope device and display device 3b are used for the system of the light source device 3 for an electronic endoscope device, and each device is configured by the specification unique to this system. In this system, light source information or the like provided by the light source device 3 for an electronic endoscope device an electronic endoscope device is obtained by the light source information acquisition device 3a for an electronic endoscope device, and is displayed on the display device 3b as requested.

Thus, conventionally, a system exists independently for each medical device, each system is configured by a unique specification and in each system, data is transmitted/received based on a unique specification.

In an endoscopic inspection, more particularly in the inspection of the coelom in a curved shape, such as the large intestine, the small intestine or the like, an endoscope shape detection device is used since it is necessary to insert an endoscope while checking the location of its tip. The endoscope shape detection device can calculate the shape of an endoscope inserted in a body part to be examined and display it on a monitor connected to the endoscope shape detection device via a video processor (see, for example, Japanese Patent Application Nos. 2001-46320 and 2000-93386).

The endoscope shape detection device receives magnetism emitted from ten and several magnetic coils built in the insertion part of an endoscope by an antenna provided at the center of the endoscope shape detection device main body. The position of each receiving coil is calculated by a computer and the three-dimensional position of each coil is connected by a smooth curved line. Then, furthermore, a graphics treatment is applied in order to make its shape more easily seen and is displayed on a monitor (see, for example, Japanese Patent Application No. 11-325810).

In the endoscope shape detection device, a marker including a coil for generating magnetism is disposed around the entrance of the endoscope, which is close to a patient. The antenna provided at the center of the endoscope shape detection device main body reads magnetism emitted by a marker, and as shown in FIG. 2, the location of a marker 91 is displayed as the starting point of the insertion of an endoscope 92 on a monitor 93. Thus, the endoscope shape detection device can check the insertion position of the endoscope by the marker. If the location of the tip of the endoscope is detected based on the position of a coil most closely built in the tip of the endoscope, of the coils built in the insertion part of the endoscope, a length between the location of the marker and the tip of the endoscope, that is the insertion length of the endoscope can be detected. As shown by a point emphasized by encircling in FIG. 2, this insertion length of the endoscope is displayed with insertion shape of the endoscope, for example, in units of centimeter on the monitor 93 of an endoscope shape detection device 94.

Conventionally, when writing an endoscopic inspection report, the copy of the observation image of a morbid part is attached to a paper carte on which the body part to be inspected is sketched. In this case, firstly if a morbid part is detected at the time of endoscopic inspection, the insertion length is, for example, recorded and stored. If the insertion length of the endoscope in the case where the morbid part is detected is known, how far the morbid part is away from the entrance of the body part to be inspected can be roughly determined based on the standard data, such as the shape, size or the like, of the body part to be inspected. Thus, when writing an inspection report after the completion of the inspection, the rough location of the morbid part is marked on the sketch. By connecting the marked point and the morbid part on the copied observation image by a line, the body part of the attached observation image can be detected. Thus, using the insertion length data of the endoscope up to the morbid part, a report is issued after the inspection.

Recently, an endoscopic inspection in which the coelom or the lumen of internal organs are directly observed by inserting the slim tip of a scope in the coelom or the like of a body part to be inspected has become popular.

An endoscopic image filing system capable of recording/storing an endoscopic image shot by an endoscope device in a server connected via a network and extracting this image as requested is widely used.

In the endoscopic image filing system, when endoscope inspector pushes a switch provided in the scope part of the endoscope device, such as a release switch, the medical image storage device connected to the endoscope device extracts an endoscopic image displayed on the monitor, patient information and inspection information are attached to it and is recorded and stored in the server via the network.

For example, in the endoscopic image filing system disclosed by Japanese Patent Application No. 7-141498, an endoscopic image shot by the endoscopic observation device is transmitted to a file server via a network and is recorded/stored in the server after the medical image storage device applies a data compression process to it.

Generally a physician or surgeon in charge of a patient requests an endoscopic inspector to conduct an endoscopic inspection.

Although the endoscopic inspector makes an inspection report after the completion of the requested inspection, an image file is attached to this inspection report.

The endoscopic inspector makes a report while calling up all images stored in the file server from a terminal connected to the file server via a network, displaying them on a screen and selecting an image to be used for the report.

Although in one endoscopic inspection, usually 40-100 pieces of images are shot, points to be shot generally fixed and some images with low importance are also included. All the shot images are not used for the report, and only a particularly important image, such as the image of a body part in which a polyp is detected is used. In this case, after all the images are taken into the terminal once, they are outputted on a screen or by a printer, and their contents are sequentially checked based on his/her memory.

### SUMMARY OF THE INVENTION

One aspect of the present invention is a communication method of a storage device for recording medical information. The communication method comprises receiving information from a plurality of devices for providing at least one of setting information, status information, inspection image information and patient information, and transmitting at least one of the setting information and status information or information obtained by processing the information, via a network, in addition to the inspection image information and patient information.

Another aspect of the present invention is a medical image storage device. The medical image storage device comprises an endoscopic observation image storage unit for recording endoscopic observation image data obtained by an endoscope, an endoscope insertion length storage unit for recording endoscope insertion length data indicating an insertion length of the endoscope in a body to be inspected and a data management unit for monitoring/managing endoscopic observation image data and endoscope insertion length data.

Another aspect of the present invention is a medical image storage device for recording endoscopic images shot by an endoscope. The medical image storage device comprises an image sensing unit for taking in an endoscopic image shot by the endoscope as image data according to the instruction of an operator and a marking unit for attaching information indicating that the endoscopic image taken in by the image sensing unit is selected, to the endoscopic image, according to the instruction of the operator.

### BRIEF DESCRIPTION OF THE DRAWINGS

The present invention will be more apparent if the following detailed description and drawings are referenced together.

FIG. 1 shows one example of the conventional medical device and its peripheral devices.

FIG. 2 shows an example of the image the endoscope insertion shape of which is displayed on the monitor of the endoscope insertion shape detection device.

FIG. 3 shows one example of the system including the image storage device of the first preferred embodiment.

FIG. 4 shows one example of the system including the conventional image storage device.

FIG. 5 shows one example of the system including the image storage device of the second preferred embodiment.

FIG. 6 shows one example of the system including the conventional image storage device.

FIG. 7 shows one example of the system including the image storage device of the third preferred embodiment.

FIG. 8 shows one example of the conventional system including a network system operated according to a unique communication protocol and a network system operated according to a DICOM protocol.

FIG. 9 shows one example of the system of the fourth preferred embodiment including a network system operated according to a unique communication protocol and a network system operated according to a DICOM protocol.

FIG. 10 explains one example of the conventional maintenance management work.

FIG. 11 shows one example of the system including the image storage device of the fifth preferred embodiment.

FIG. 12 shows one example of the system including the image storage device of the sixth preferred embodiment.

FIG. 13 explains the conventional record of history data (No. 1).

FIG. 14 explains the conventional record of history data (No. 2).

FIG. 15 explains the conventional record of history data (No. 3).

FIG. 16 shows one example of the system including the image storage device of the seventh preferred embodiment.

FIG. 17 shows one example of the system including the conventional image storage device.

FIG. 18 shows one example of the system including the image storage device of the eighth preferred embodiment.

FIG. 19 shows one example of the system including the image storage device of the ninth preferred embodiment.

FIG. 20 shows one example of information stored in an external storage.

FIG. 21 shows one example of the system including the image storage device of the tenth preferred embodiment.

FIG. 22 shows one example of the system including the image storage device of the eleventh preferred embodiment.

FIG. 23 shows one example of the system including the image storage device of the twelfth preferred embodiment.

FIG. 24 shows an example of the configuration of an image storage device for transferring original image information for each plane of R, G and B.

FIG. 25 shows the system configuration of the thirteenth preferred embodiment.

FIG. 26 is a flowchart showing the process of linking a sketch and the observation image of an endoscope.

FIG. 27 is an example of the image showing the sketch of a report.

FIG. 28 typically shows the relationship between the medical image storage device, electronic endoscope device and endoscope insertion shape detection device in the fourteenth preferred embodiment.

FIG. 29 shows the basic configuration of an information storage device in a data processing part.

FIG. 30 shows the configuration of the endoscopic image filing system in the fifteenth preferred embodiment.

FIG. 31 shows an example of the configuration of the image storage device.

FIG. 32 is a flowchart showing the operational process of the image storage device at the time of endoscopic inspection.

FIG. 33 shows an example of the procedure of issuing a report.

FIG. 34 shows an example of the image selection screen (No. 1).

FIG. 35 shows an example of the image selection screen (No. 2).

FIG. 36 shows an example of the report.

FIG. 37 shows an example of the structure of the image file.

FIG. 38 shows an example of the structure of the file in the server.

### DESCRIPTION OF THE PREFERRED EMBODIMENT

The preferred embodiments of the present invention are described below with reference to the drawings.

### The First Preferred Embodiment

Firstly, the first preferred embodiment is described.

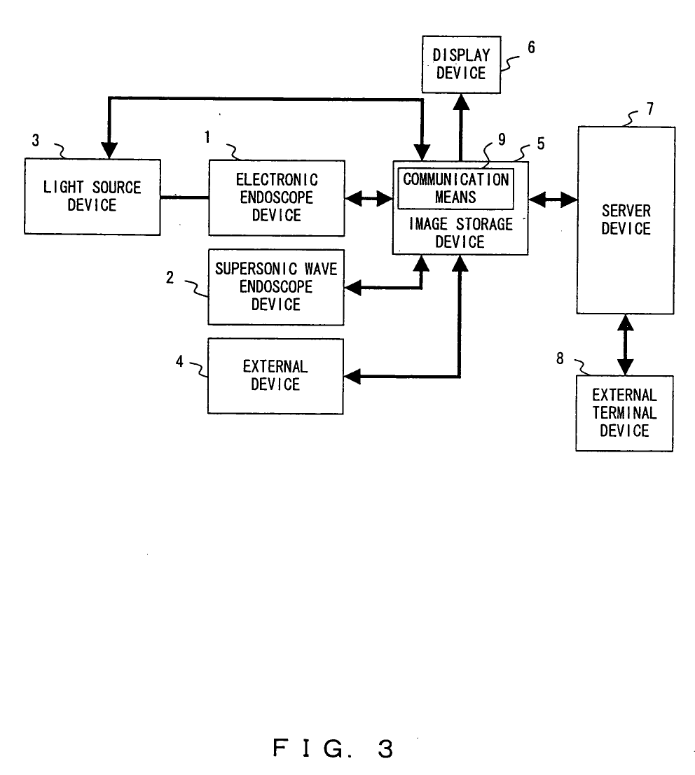

FIG. 3 shows one example of the system including the image storage device of the first preferred embodiment.

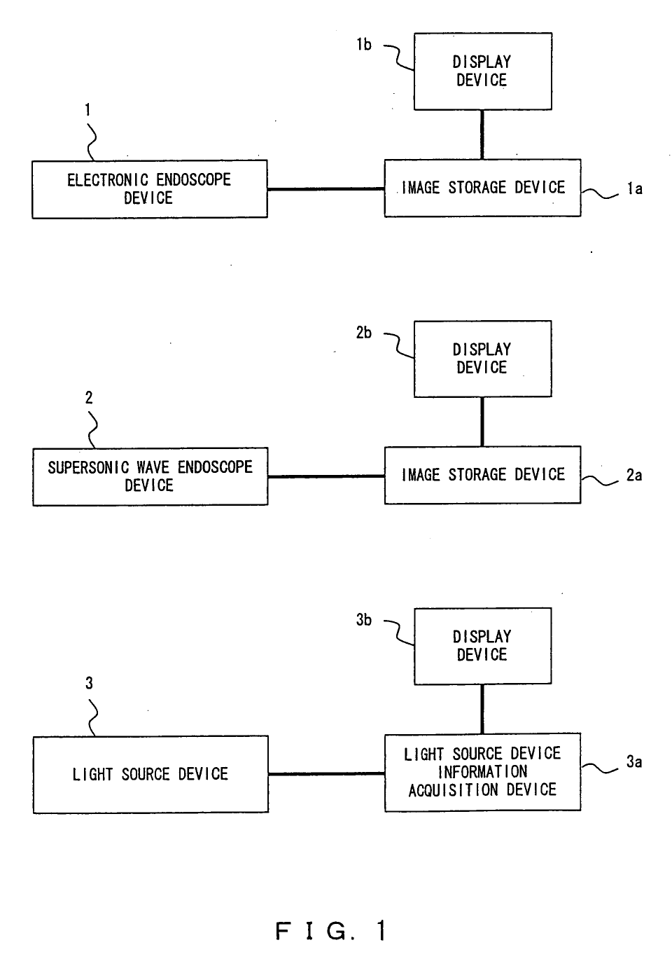

As shown in FIG. 3, each of an electronic endoscope device 1, a light source device 3 for the electronic endoscope device for supplying the device 1 with light source, a supersonic wave endoscope device 2 and an external device 4 is connected to an image storage device 5. The devices 1 through 3 are the same as those shown in FIG. 1. Each of the devices 1 through 3 and device 4 transmits/receives data, based on their unique specifications. The external device 4 can be, for example, a magnetic card reader for reading the data of an ID card possessed by a patient, or a device used together with an inspection device or the like, other than the electronic endoscope device 1 and supersonic wave endoscope device 2, and is not limited to a specific device.

The image storage device 5 comprises a communication means 9 capable of transmitting/receiving data even when located between the devices 1 through 4 for transmitting/receiving data, based on such a unique specification. The image storage device 5 can collectively or separately obtain a plurality of pieces of information provided by these devices 1 through 4, and record and processes the information. Specifically, the image storage device 5 collectively or separately obtains an inspection image and patient information which are provided by the electronic endoscope device 1, the setting information and status information of the device 1 which are provided by the device 1, an inspection image and patient information which are provided by the supersonic wave endoscope device 2, the setting information and status information of the device 2 which are provided by the device 2, light source information provided by the light source device 3 for the electronic endoscope device and predetermined information provided by the external device 4, and records and process the obtained information. The image storage device 5 also transmits setting modification instructing information to one of corresponding devices 1 through 4, according to a setting modification instruction or the like to one of these devices 1 through 4, and modifies the setting of one of the corresponding devices 1 through 4.

The image storage device 5 is also connected to a display device 6, and displays obtained information or information obtained by processing it.

The image storage device 5 is also connected to a server device 7, an external terminal device 8 and the like via a network, and transmits the obtained information or information obtained by processing it to the server device 7. Thus, information obtained by the image storage device or the like is stored in the server device 7, and a plurality of the obtained information is stored in the server device 7, and can be collectively managed. The external terminal device 8 reads the information stored in the server device 7 and displays on a display, which is not shown in FIG. 3, as requested. Thus, information obtained by the image storage device 5 or the like can be read via the external terminal device 8.

As described above, according to the present invention, one image storage device can obtains plurality of pieces of information provided by a variety of devices for transmitting/receiving data, based on each unique specification. Therefore, there is no need to install a corresponding special device for each device, thereby realizing a favorable system capable of saving cost and space as a whole.

Since one image storage device can collectively or separately a plurality of pieces of information provided by a variety of devices, information can be easily managed, and the relationship between the devices can be easily obtained.

Furthermore, since one image storage device can set a variety of devices, the devices can be easily managed, and the environment setting of the entire system can be simplified.

In the image storage device 5 of this preferred embodiment, the synthesis of devices for outputting obtained information is not limited to four types of the devices 1 through 4, and another synthesis of the types and numbers of devices is also possible.

In the image storage device 5 of this preferred embodiment, data can also be wirelessly transmitted/received between connected devices.

### The Second Preferred Embodiment

Next, the second preferred embodiment of the present invention is described.

As described above with reference to FIG. 1, conventionally, since each system is configured based on a unique specification, in the image storage device of each system (for example, the image storage device 1a shown in FIG. 1), obtained image information is recorded in a unique image recording format. Therefore, a device for reproducing recorded image information also requires a special device corresponding to the image recording format, and only a facility with the same system can read the image information, which is a problem.

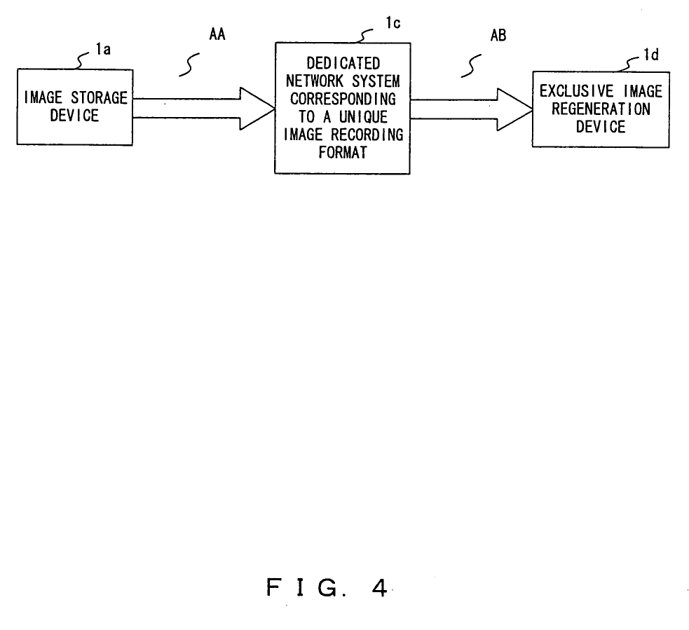

FIG. 4 shows one example of the system including the conventional image storage device.

In FIG. 4, an image storage device (here it is 1a shown in FIG. 1) is connected to a dedicated network system 1c corresponding to a unique image recording format, to which an exclusive image regeneration device 1d corresponding the image recording format is connected. In such a system, image information recorded in the unique image recording format by the image storage device 1a is stored in a server device, which is not shown in FIG. 4, included on the dedicated network system 1c (arrow mark AA), is read as requested (arrow mark AB) and is regenerated by the exclusive image regeneration device 1d.

However, lots of systems corresponding to JPEG (Joint Photograph Expert Group), which is a general image recording format, exist, and lots of devices provided with a function to regenerate image information in a JPEG format also exist. Many of personal computers provided with an operating system (OS), such as windows (trademark) widely used or the like, are compatible with this JPEG format.

Therefore, in the image storage device of this preferred embodiment, obtained image information (such as inspection image information) can be recorded in either a unique image recording format or a JPEG format, or both of them.

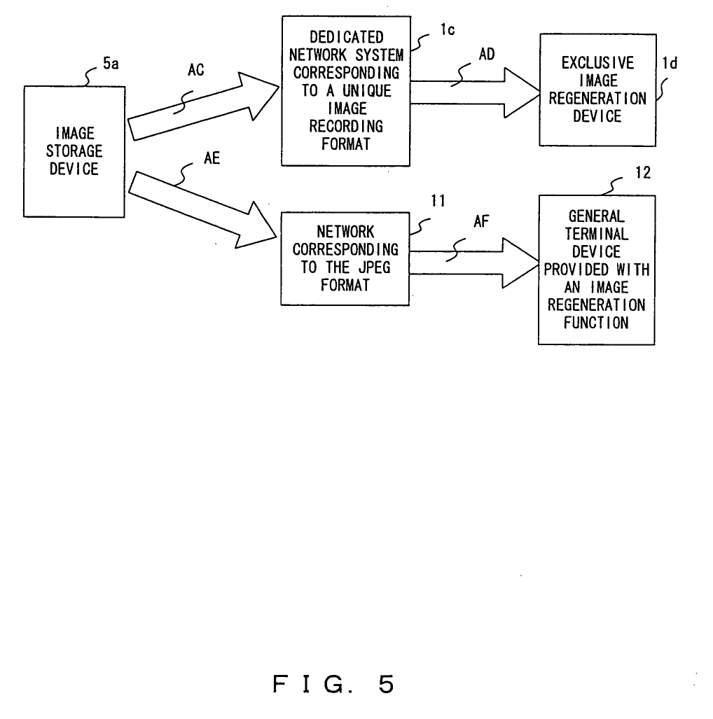

FIG. 5 shows one example of the system including the image storage device of this preferred embodiment.

In FIG. 5, an image storage device 5a is connected to a dedicated network 1c corresponding to a unique image recording format and a network 11 corresponding to the JPEG format. To the network system 1c, an exclusive image regeneration device 1d corresponding to the unique image recording format is connected, and to the network system 11, a general terminal device 12 provided with an image regeneration function corresponding to the JPEG format (such as a personal computer provided with the Windows OS, etc.) is connected.

The image storage device 5a records obtained image information in either the unique image recording format or the JPEG format, or both of the image recording formats, according to the selection setting of the image recording format. The other configuration of the image storage device 5a is the same as the image storage device 5 of the first preferred embodiment.

If in a system with such a configuration, image information obtained by the image storage device 5a is read using the exclusive image regeneration device 1d corresponding to the unique image recording format, the image storage device 5a is set in such a way that the obtained image information can be recorded in the unique image recording format (or the obtained image information can be recorded in both of the image recording formats). Thus, if image information is obtained by the image storage device 5a, the image information is recorded in the unique image recording format. Then, the image information recorded thus is transmitted to and stored in a server device, which is not shown in FIG. 5, included in the dedicated network system 1c corresponding to the unique image recording format (arrowmark AC), is read as requested (arrowmark AD) and is displayed on the image regeneration device 1d.

If the image information obtained by the image storage device 5a is read using the general terminal device 12 corresponding to the JPEG format, the image storage device 5a is set in such a way that the obtained image information can be set in the JPEG format (or the obtained image information can be recorded in both of the image recording formats). Thus, if image information is obtained by the image storage device 5a, the image information is recorded in the JPEG format. Then, the image information recorded thus is transmitted to and stored in a server device, which is not shown in FIG. 5, included in the dedicated network system 11 corresponding to the JPEG format (arrow mark AE), is read as requested (arrow mark AF) and is displayed on the terminal device 12.

If it is set in such a way that the obtained image information can be recorded in both of the image recording formats, when the image information is obtained by the image storage device 5a, the image information is recorded in either of the unique and JPEG image recording formats, and the image information recorded in the unique image recording format is transmitted to and stored in the server device included in the dedicated network system 1c corresponding to the unique image recording format (arrow mark AC). Then, the image information recorded in the JPEG format is transmitted and stored in the network system 11 corresponding to the JPEG format (arrow mark AE).

As described above, according to this preferred embodiment, since obtained image information can be recorded in two different images recording formats, using one image storage device. Therefore, there is no need to provide an image storage device for each corresponding image recording format as conventionally, thereby realizing a favorable system capable saving cost and space as a whole.

If a general terminal device provided with a regeneration function corresponding to the JPEG format exists, there is no need for an exclusive image regeneration device corresponding to the unique image recording format, thereby easily realizing the distribution and secondary utilization of image information.

Since it can be recorded in the JPEG format, which is a general image recording format, a regeneration terminal device can also be easily obtained.

Furthermore, if the use of image information is separated, it is effective. For example, if highly secret image information can be recorded in the unique image recording format, and low secret image information (no secret image information) can be recorded in the JPEG format.

Although in the image storage device of this preferred embodiment, obtained image information is recorded in both of the unique and JPEG image recording formats, it can also be recorded in another synthesis of two different types of image recording formats. Alternatively, it can be recorded in the synthesis of three or more types of image recording formats. In this case, for example, it can be recorded in at least one image recording format selected from the three or more types of image recording formats.

### The Third Preferred Embodiment

Next, the third preferred embodiment is described.

As described above with reference to FIG. 1, since conventionally, each system is configured based on a unique specification, and in the image storage device of each system (for example, the image storage device la shown in FIG. 1), obtained image information is recorded in a unique image recording format and the image information recorded in the unique image recording format is used in the system.

However, recently, DICOM (Digital Imaging and Communications in Medicine) has been becoming popular as a standard for medical images and communication, and a system corresponding to DICOM also exists.

In such a situation, it is required that image information recorded in the above-mentioned unique image recording format can also be used even in a system corresponding to DICOM.

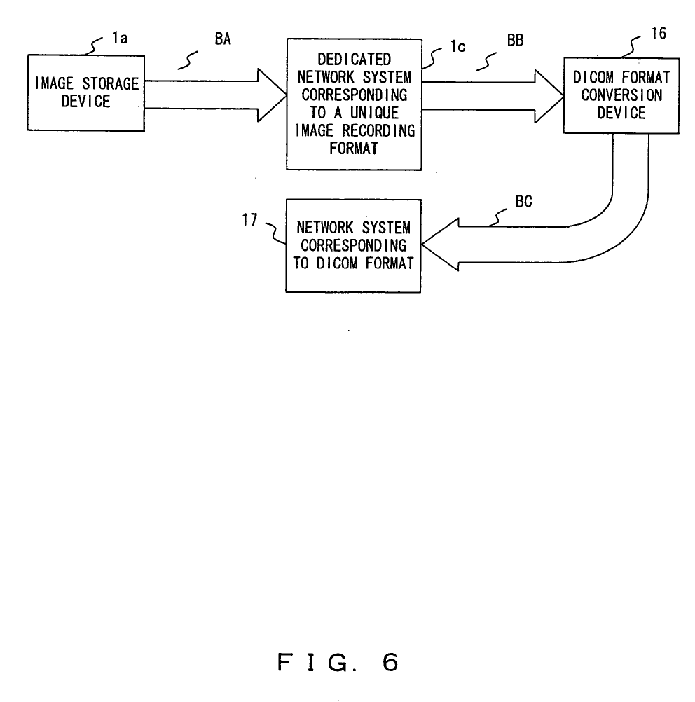

FIG. 6 shows one example of the system including the conventional image storage device.

In FIG. 6, an image storage device (here it is la shown in FIG. 1) is connected to the dedicated network system 1c corresponding to a unique image recording format, which is connected to a network system 17 corresponding to an image recording format which can be handled in a system corresponding to DICOM (herein after called a “DICOM format”) via a DICOM format conversion device 16 for converting image information in the unique image recording format into image information in the DICOM format. In such a system, the image information recorded in the unique image recording format by the image storage device 1a is network-transferred to and stored in a server device, which is not shown in FIG. 6, included in the dedicated network system 1c (arrow mark BA) and is read as requested (network-transferred) (arrow mark BB). Then, the image information is converted into image information in the DICOM format by the DICOM format conversion device 16 and is transferred to and stored in a server device, which is not shown in FIG. 6, included in the network system 17 corresponding to the DICOM format (arrow mark BC). Then, the image information is read and used in the network system 17, as requested.

Thus, conventionally, in order to use image information recorded in the unique image recording format in the network system corresponding to the DICOM format, a conversion device for convert it into the image information in the DICOM format is needed, thereby increasing the size of the system as a whole or complicating the system.

Therefore, in the image storage device of this preferred embodiment, obtained image information (such as inspection image information) can be recorded in either the unique or DICOM image recording formats, or both of the image recording formats.

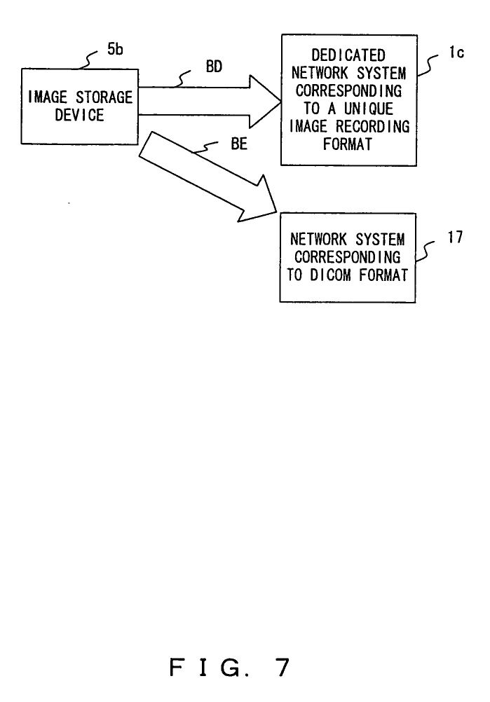

FIG. 7 shows one example of the system including the image storage device of this preferred embodiment.

In FIG. 7, an image storage device 5b is connected to the dedicated network system 1c corresponding to the unique image recording format and the network system 17 corresponding o the DICOM format.

The image storage device 5b records obtains image information in either the unique or DICOM image recording formats, or both of the image recording formats. The other configuration of the image storage device 5b is the same as in the image storage device 5 of the first preferred embodiment.

In a system with such a configuration, if image information obtained by the image storage device 5b in a dedicated network system corresponding to a unique image recording format is used, the image storage device 5b is set in such a way that the obtained image information can be set in the unique image recording format (or the obtained image information can be recorded in both of the image recording formats). Thus, if image information is obtained by the image storage device 5b, the image information is recorded in the unique image recording format. Then, the image information recorded thus is transmitted to and stored in a server device, which is not shown in FIG. 6, included in the dedicated network system 1c corresponding to the unique image recording format (arrow mark BD), is read and used as requested.

If the image information is obtained by the image storage device 5b in the network system 17 corresponding to the DICOM format, the image storage device 5b is set in such a way that the obtained image information can be recorded in the DICOM format (or the obtained image information can be recorded in both of the image recording formats). Thus, when the image information is obtained by the image storage device 5b, the image information is recorded in the DICOM format. Then, the image information recorded thus is transmitted to and stored in the server device, which is not shown in FIG. 6, included in the network system 17 corresponding to the DICOM format (arrow mark BE) and is read and used as requested.

If the obtained image information is set in such a way as to be recorded in both of the image recording formats, when the image information is obtained by the image storage device 5b, the image information is recorded in both of the unique and DICOM image recording formats, and the image information recorded in the unique image recording format is transmitted to and stored in a server device included in the network system 1c corresponding to the unique image recording format (arrow mark BD). Then, the image information recorded in the DICOM format is transmitted to and stored in a sever device included in the network system 17 corresponding to the DICOM format (arrow mark BE).

As described abode, according to this preferred embodiment, one image storage device can record obtained image information in two different types of image recording formats. Therefore, the obtained image information can be connected to network systems corresponding to different image recording formats without the provision of a device for converting the obtained image information into a different image recording format as conventionally, thereby realizing a favorable system capable of saving cost and space as a whole.

Since there is no need to transfer the obtained image information to a conversion device as conventionally, labor and cost needed for the transfer can also be reduced.

Since image information recorded by an image storage device can be simultaneously handled in two network systems the corresponding image recording formats of which are different, for example, the efficiency of the reading work of image information can be improved.

Although the image storage device of this preferred embodiment records obtained image information in a unique and DICOM image recording formats, the obtained image information can also be recorded in another synthesis of two different types of image recording formats. Alternatively, the obtained image information can be recorded in the synthesis of three or more different types of image recording formats. In this case, the obtained image information can be recorded in at least one image recording format selected from the three or more types of image recording formats.

### The Fourth Preferred Embodiment

Next, the fourth preferred embodiment of the present invention is described.

As described above with reference to FIG. 1, since conventionally, each system is configured based on a unique specification, there is a system in which data is transmitted/received using a unique communication protocol in them.

However, recently, DICOM has been becoming popular as a standard for medical images and communication, and a system in which data is transmitted/received using a communication protocol for DICOM (hereinafter called a “DICOM protocol”) is also exists.

In such a situation, it becomes necessary to also handle information handled in a system which is operated according to the above-mentioned unique communication protocol in a system according to the DICOM protocol.

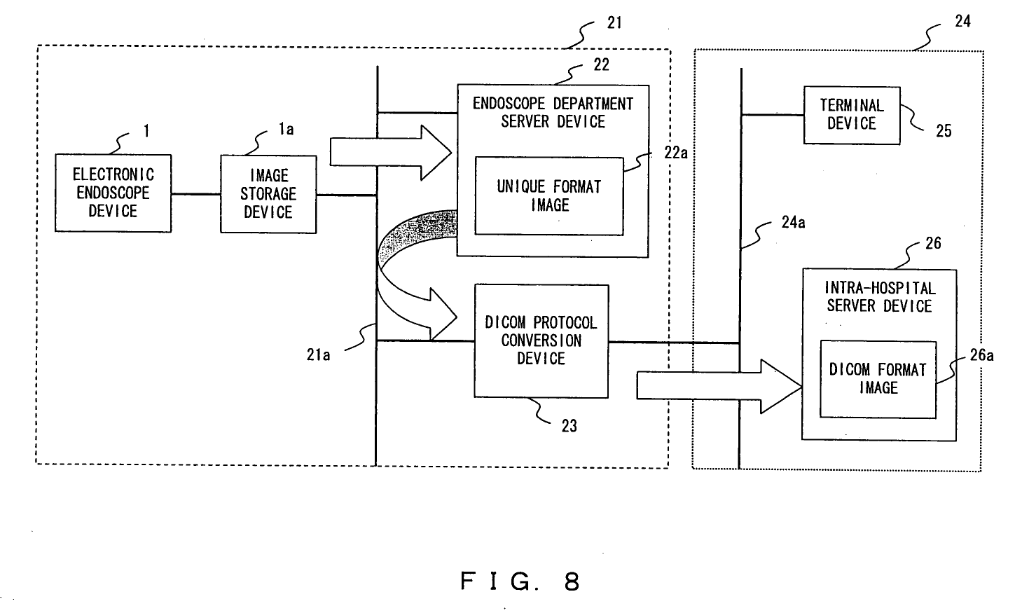

FIG. 8 shows one example of the conventional system including a network system operated according to the unique communication protocol and a network system operated according to the DICOM protocol.

In FIG. 8, an image storage device (here it is 1a shown in FIG. 1) is connected to an endoscope device (here it is 1 shown in FIG. 1) as a component in an intra-endoscope department network system 21, and an endoscope department server device 22, a DICOM protocol conversion device 23 and the like are also connected to the device 1a via the network 21a operated by a unique communication protocol. The DICOM protocol conversion device 23 is connected to a network 24a operated according to the DICOM protocol in an intra-hospital network system 24, and a terminal device 25 and intra-hospital server device 26 and the like are also connected to the device 23 via the network 24a operated according to the DICOM protocol.

In a system with such a configuration, if image information provided by the endoscope device 1 is used in the intra-hospital network system 24, the following data is transmitted/received. Firstly, when the image information provided by the endoscope device 1 is obtained by the image storage device 1a, the image information is temporarily stored in the endoscope department server device 22 via the network 21a operated according to the unique communication protocol (storage of a unique format image 22a). Then, the stored image information is read and is converted into image information according to the DICOM protocol by the DICOM protocol conversion device 23. Then, the converted image information is stored in the intra-hospital server device 26 via the network 24a an operated according to the DICOM protocol (storage of a DICOM format image 26a). Then, the image information is read and used by the terminal device 25 or the like, as requested, and the image information is viewed.

As described above, conventionally, in order to handle information handled in a system according to the unique communication protocol in a system operated according to the DICOM protocol, a communication protocol conversion device is necessary, thereby increasing the size of a system as a whole and complicate the system.

Therefore, in the image storage device of this preferred embodiment, obtained image information can be converted into and outputted (transmitted) as either image information according to the unique communication protocol or image information according to the DICOM protocol, or image information according to both the communication protocols.

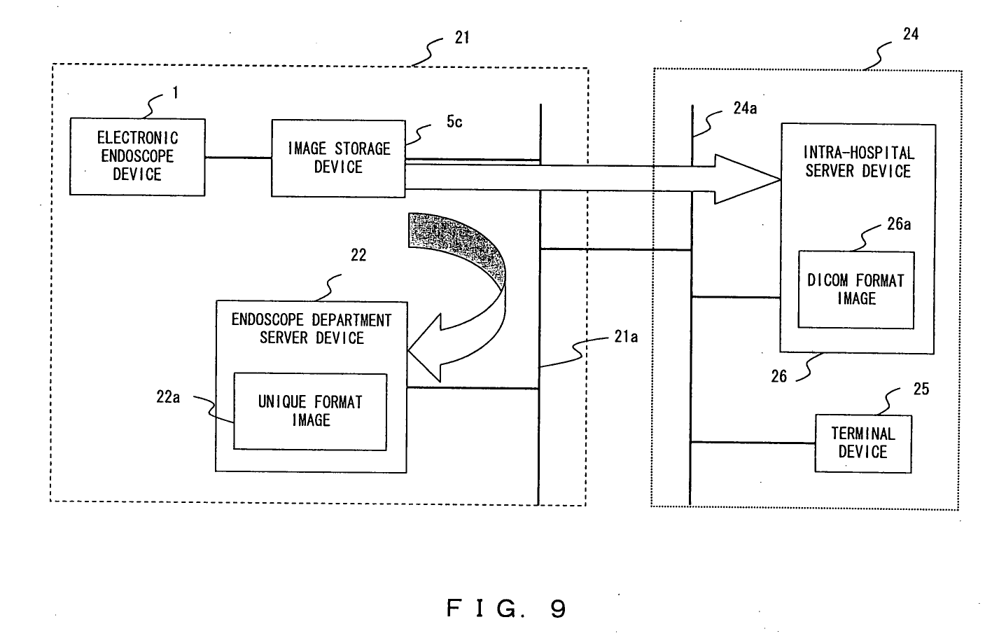

FIG. 9 shows one example of the system of this preferred embodiment including a network system operated according to the unique communication protocol and a network system operated according to the DICOM protocol, which corresponds to FIG. 8.

In FIG. 9, as a component in an intra-endoscope department network system 21, an image storage device 5c is connected to the endoscope device 1, and the endoscope server device 22 and the like are also connected to the device 5c via the network 21a operated according to the unique communication protocol. The network 21a operated according to the unique communication protocol is connected to the network 24a operated according to the DICOM protocol in the intra-hospital network system 24. The configuration of the intra-hospital network system 24 is already described above.

When outputting (transmitting) image information obtained from the endoscope device 1 according to the selection setting of a communication protocol, the image storage device 5c converts the image information into either image information according to the unique communication protocol or image information according to the DICOM protocol, or image information according to both the communication protocols and outputs (transmits) it. The other configuration of the image storage device 5c is the same as the image storage device 5 of the first preferred embodiment.

In a system with such a configuration, if the image information obtained from the endoscope device 1 is used in the network system 24a operated according to the DICOM protocol, the image storage device 5c is set in such a way as to convert the obtained image information into image information according to the DICOM protocol and to output (transmit) it (or to convert the obtained image information into image information according to both the communication protocols and to output (transmit) it). Thus, when the image storage device 5c obtains the image information from the endoscope device 1, the image information is converted into and outputted (transmitted) as image information according to the DICOM protocol and is stored in the intra-hospital server device 26 via the network 21a operated according to the unique communication protocol and the network 24a operated according to the DICOM protocol (storage of a DICOM format image 26a). Then, it is read and used, as requested.

If the image information obtained from the endoscope device 1 is used in the network system 21 operated according to the unique communication protocol, the image storage device 5c is set in such a way as to convert the obtained image information into image information according to the unique communication protocol and to output (transmit) it (or to convert the obtained image information into image information according to both the communication protocols and to output (transmit) it). Thus, when the image storage device 5c obtains the image information from the endoscope device 1, the image information is converted into and outputted (transmitted) as image information according to the unique communication protocol and is stored in the endoscope department server device 22 via the network 21a operated according to the unique communication protocol (storage of a unique format image 22a), and is read and used, as requested.

If the obtained image information is set in such away as to be converted into and outputted (transmitted) as image information according to both the communication protocols, when the image storage device 5c obtains the image information from the endoscope device 1, the image information is converted into and outputted (transmitted) as image information according to the DICOM protocol and simultaneously is converted into and outputted (transmitted) as image information according to the unique communication protocol. Then, the image information according to the DICOM protocol is stored in the intra-hospital server device 26 (storage of a DICOM format image 26a), and image information according to the unique communication protocol is stored in the endoscope server device 22 (storage of a unique format image 22a).

As described above, according to this preferred embodiment, since one image storage device can output (transmit) obtained image information in two different types of communication protocols, the image storage device can be connected to network systems operated according to different communication protocols without providing a communication protocol conversion device as conventionally, thereby realizing a favorable system capable of saving cost and space as a whole.

Since there is no need to transfer image information to a conversion device as conventionally, labor and cost needed for the transfer can also be reduced.

Since image information obtained by an image storage device can be simultaneously handled in two network systems each operated according to a different communication protocol, for example, the efficiency of the reading work of image information can also be improved.

Although the image storage device of this preferred embodiment converts obtained image information into image information according to the unique communication protocol and the DICOM protocol and to output (transmit) it, the image information can also be converted into and outputted (transmitted) as image information according to another synthesis of two different types of communication protocols. Alternatively, the image information can be converted into and outputted (transmitted) as image information according to the synthesis of three or more different types of communication protocols. In this case, for example, the obtained image information can also be converted into and outputted (transmitted) as image information according to at least one communication protocol selected from the three or more different types of communication protocols.

Although in the image storage device of this preferred embodiment, information to be outputted (transmitted) is image information, it can also be an inspection image or patient information.

### The Fifth Preferred Embodiment

Next, the fifth preferred embodiment of the present invention is described.

As described above with reference to FIG. 1, since conventionally, each system is configured based on a unique specification, the device maintenance work of each system is separately conducted. For example, some image storage devices are provided with a function to record a variety of information, such as the setting information, status information or the like of the device and to utilize them for its maintenance or the like in an image storage device (such as the image storage device 5 shown in FIG. 1). A device whose function can be realized by a program (software) is also proposed. However, when doing the maintenance work of an image storage device, such as the acquisition of the setting information, etc., the update of the program and the like, information must be directly extracted from the device itself to be maintained or the control program of the device itself must be directly rewritten, labor for collecting a device for its maintenance, labor for visiting a place where a device is installed or labor for connecting a maintenance device or opening/shutting the cabinet of a device is required. Therefore, its maintenance cannot be easily done. As shown in FIG. 10, in order to do the maintenance works 99, 100 ad 101 of a plurality of image storage devices 96, 97 and 98 at a time, their maintenance works must be separately done. Therefore, it takes much labor, and there is a possibility that an operation mistake, such as a wrong setting or the incomplete update of the program may occur.

Therefore, in the image storage device of this preferred embodiment, the maintenance work can be done from the maintenance device connected to a network in order to reduce the load of such a maintenance work.

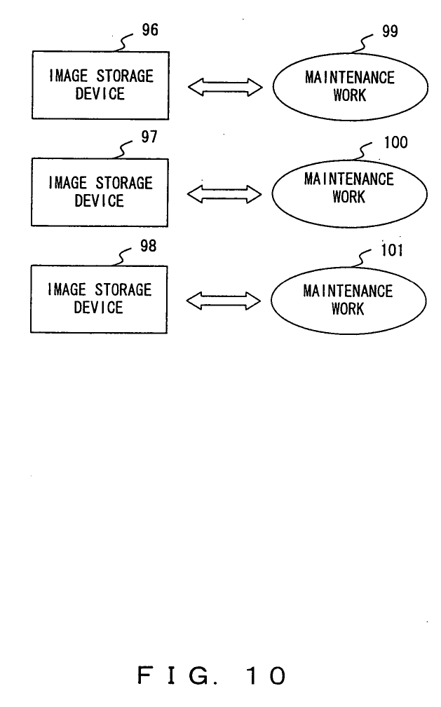

FIG. 11 shows one example of the system including the image storage device of this preferred embodiment.

In FIG. 11, image storage devices 5d and 5e are connected to a maintenance device (terminal device) 31 via a network.

Each of the image storage devices 5d and 5e outputs information (setting information, status information, etc.) as a history and the like to the maintenance device 31, modifies the setting of the relevant device, based on the setting information outputted from the maintenance device 31, updates the program of the relevant device to an update program outputted from the maintenance device 31 and so on. The other configuration of each of the image storage device 5d and 5e is the same as in the image storage device 5 of the first preferred embodiment.

In a system with such a configuration, when doing the maintenance of the image storage devices 5d and 5e simultaneously, the maintenance device 31 obtains and displays a plurality of pieces of information 102 and 103, such as a history or the like from each of the image storage devices 5d and 5e via a network. For example, the respective statuses of the image storage devices 5d and 5e are displayed (31a shown in FIG. 11). Corresponding setting information outputted to each of the image storage devices 5d and 5e. In each of the image storage devices 5d and 5e, the setting of the relevant device is modified based on the setting information. The maintenance device 31 stores the update program (31b shown in FIG. 11) of each of the image storage devices 5d and 5e, and each of the update programs is outputted to each of the image storage devices 5d and 5e. In each of the devices 5d and 5e, the program 104 or 105 of the relevant device is updated to the update program.

Thus, the maintenance device 31 connected to the network do the respective maintenance of the image storage devices 5d and 5e simultaneously.

As described above, according to this preferred embodiment, the respective statuses and the like of all image storage devices can be obtained via a terminal device connected to a network, and the respective settings of all the image storage devices, the update of respective programs and the like can be made, thereby simplifying its maintenance work.

Since all programs can be simultaneously updated via a terminal device connected to a network, there is also no need to prepare a storage medium on which an update program is recorded, for each image storage device, which is effective in saving costs.

Since their maintenance work can be simultaneously done via a terminal device connected to a network, human operation mistakes, such as the incomplete update of a program and the like can also be reduced to a minimum level.

Although in this preferred embodiment, devices to be maintained are two image storage devices, the number and type of the devices to be maintained can also modified.

### The Sixth Preferred Embodiment

Next, the sixth preferred embodiment of the present invention is described.

This preferred embodiment aims to simplify the maintenance of an image storage device to which a plurality of devices is connected.

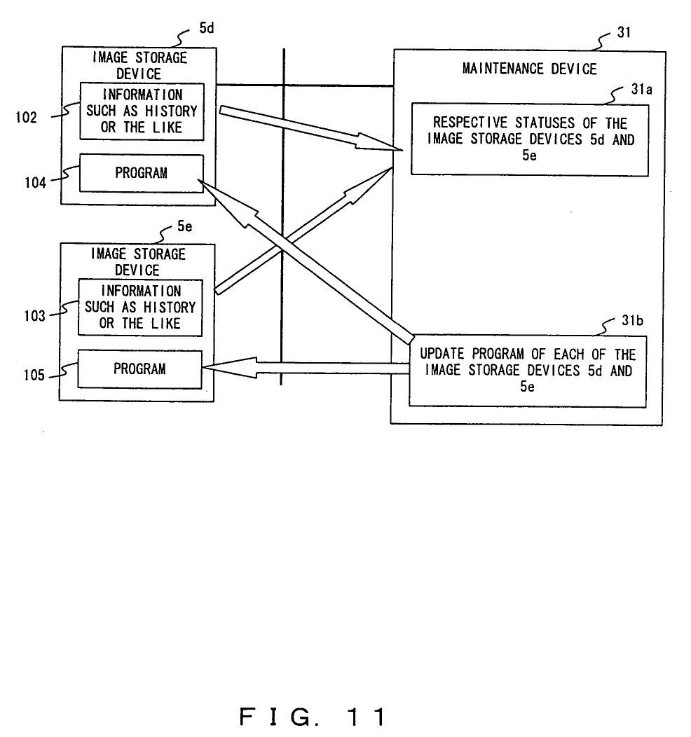

FIG. 12 shows one example of the system including the image storage device of this preferred embodiment.

In FIG. 12, external connection devices 36 and 37 are connected to an image storage device 5f as a device which is the output source of information to be obtained, and video signals and the like are outputted from each of them to the device 5f. The external connection devices 36 and 37 are, for example, the electronic endoscope device 1, supersonic wave endoscope device 2, shown in FIG. 1 or the like. To the image storage device 5f, a display device 6 for displaying a maintenance screen or the like (the same as shown in FIG. 1) is also connected.

The image storage device 5f displays the input status of each video signal on the maintenance screen of the display device 6, according to the input/no-input of a video signal outputted from each of the external connection devices 36 and 37. The image storage device 5f also records the setting information, status information and the like of the relevant device, outputted from each of the external connection devices 36 and 37 as history information 106 or the like. The other configuration of the image storage device 5f is the same as the image storage device 5 shown in FIG. 1.

In a system with such a configuration, for example, if a communication line 107 between the external connection device 36 and image storage device 5f is disconnected (broken), no video signal is inputted to the image storage device 5f from the external connection device 36, and the fact is displayed on the display device 6. For example, on the maintenance screen 108 shown in FIG. 6, “VIDEO INPUT A OFF” indicating that there is no video signal from the external connection device 36 is displayed. In this case, since video signals are normally inputted from the external connection device 37, as to the external connection device 37, “VIDEO INPUT B ON” indicating that video signals are inputted is displayed.

Thus, if any of the plurality of external connection devices connected to the image storage device fails, the failed point can be easily pinpointed without viewing by human eyes the connection with each external connection device, checking it using an inspection device or the like or obtaining and analyzing history information outputted from the image storage device or each external connection device by a variety of communication means.

As described above, according to this preferred embodiment, the check of its operation, the analysis of a failure or the like can be made without using an inspection device or the like, by displaying the input status of signals outputted from each of the plurality of devices on the maintenance screen, thereby simplifying its maintenance work and saving costs.

Although in the image storage device of this preferred embodiment, the input/no-input of video signals outputted from each external connection device is displayed on a maintenance screen, for example, a communication status with each external connection device can also be checked if necessary and the check result (whether the communication status is normal) can also be displayed on the maintenance screen.

Although two different types of devices are connected to the image storage device of this preferred embodiment as devices which are the output sources of information to be obtained, another number of different types of devices can also be connected.

### The Seventh Preferred Embodiment

Next, the seventh preferred embodiment of the present invention is described.

Some conventional image storage devices (such as the image storage device 5 shown in FIG. 1, etc.) sequentially record a variety of information, such as the operating status of a connected device, the contents of an error, and the like in the device as history data in order to obtain information, to analyze or recover a failure or the like, read the history data from the device if necessary and simplify work, such as the analysis, the recovery of the failure or the like.

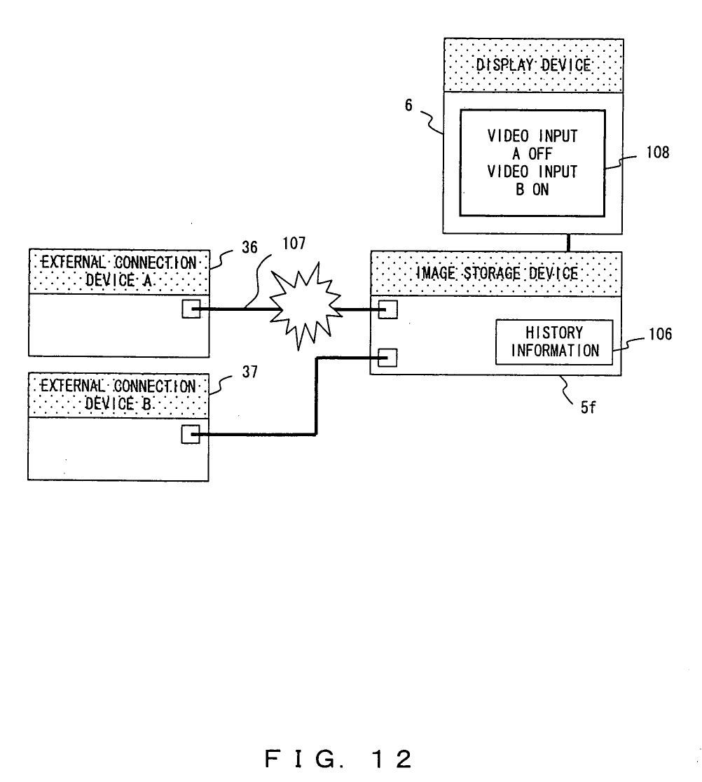

However, in such a image storage device, if a specific amount of history data is already recorded and there is no free memory capacity sufficient to record new history data, in order to record the new history data, for example, a process shown in FIGS. 13, 14 or 15 is performed.

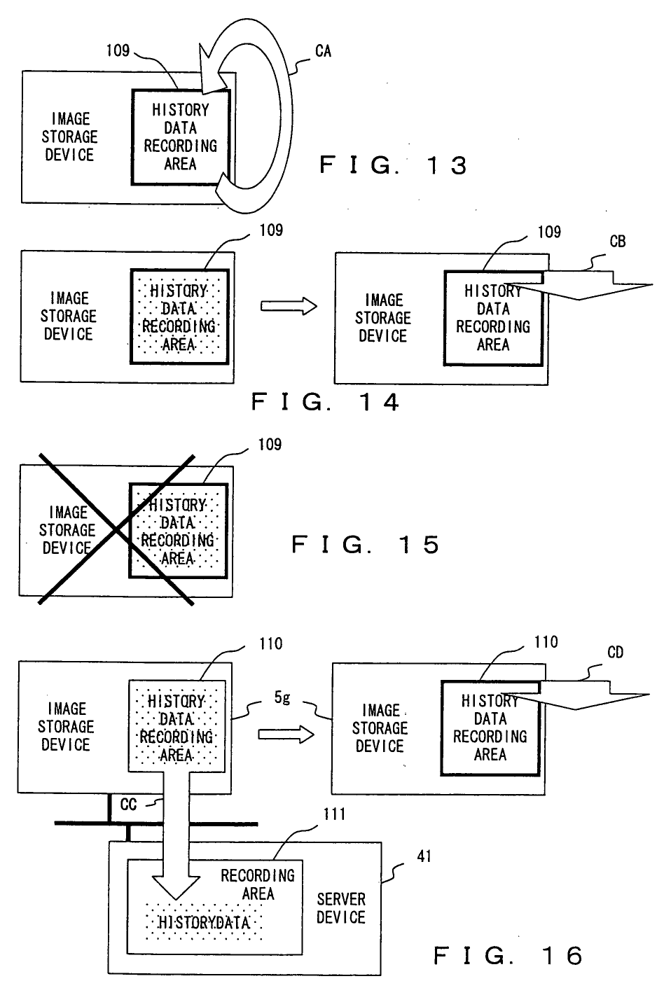

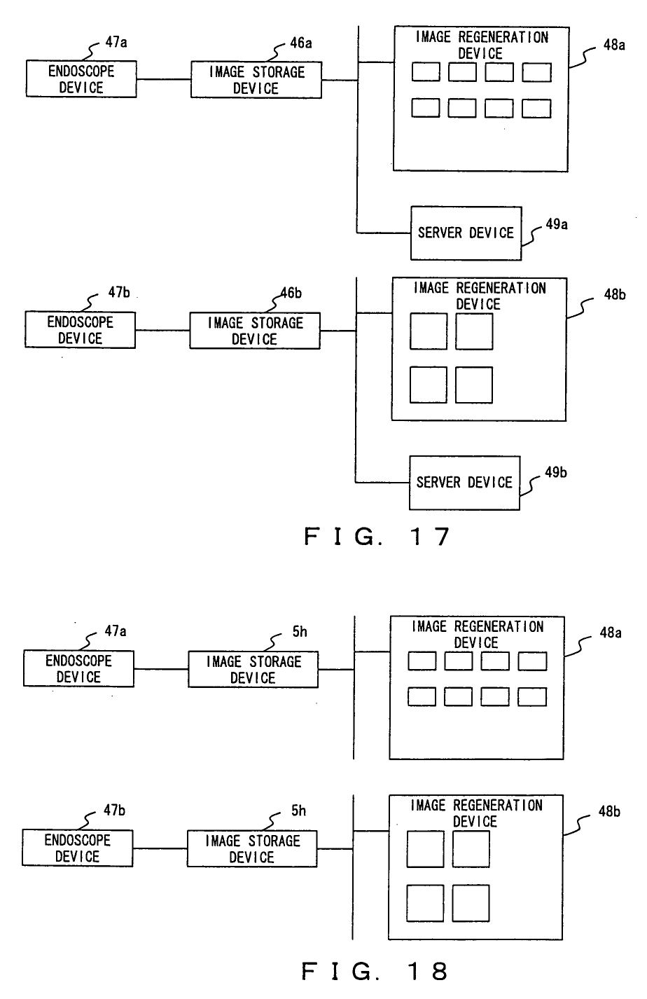

Specifically, in that case, in the image storage device, as shown in FIG. 13, such data is overwritten from the head of a history data recording area 109 (arrow mark CA). Alternatively, as shown in FIG. 14, history data already recorded on the area 109 can be all deleted and such data is recorded on the area 109 from which all data is deleted (arrow mark CB). Alternatively, as shown in FIG. 15, no operation can be made (no data is recorded).

It is difficult to record all history data in such a way, and it is also difficult to analyze a failure or detect the operation status of a device, which are problems.

Therefore, in the image storage device of this preferred embodiment, history data can be stored in a server device every time the image storage device starts, and all history data can be recorded.

FIG. 16 shows one example of the system including the image storage device of this preferred embodiment.

As shown in FIG. 16, an image storage device 5g is connected to a server device 41 via a network.

The image storage device 5g transmits history data recorded in the recording area 110 of the device 5g to the server device 41 via the network every time it is started (arrow mark CC) and records it in the history data recording area 111 of the server device 41 (history data storing area). Then, the history data recording area 110 of the image storage device 5g is released. Then, new history data is recorded in the released recording area 110 (arrow mark CD). The other configuration of the image storage device 5g is the same as the image storage device 5 of the first preferred embodiment.

As described above, according to this preferred embodiment, since history data recorded in the image storage device is recorded in a server device every time the image storage device is started, all history data can be obtained from the start of the image storage device until the current time, and the status of the image storage device can be checked from its start.

Since the history data recording area of the image storage device is released every time it is started, there is also no need for an avoidance operation or the like due to the overcapacity of the recording area.

Since history data is recorded from its current start until its next start, the status of the image storage device can also be easily checked.

### The Eighth Preferred Embodiment

Next, the eighth preferred embodiment of the present invention is described.

A plurality of systems including the conventional image storage device for generating and displaying a plurality of thumbnails which become indexes for viewing images, based on inputted video signals and displaying a original image corresponding to a thumbnail selected and specified from them exist.

In these systems, since as described with reference to FIG. 1, each system exists independently, the size of a required thumbnail varies depending on the synthesis of a device for outputting video signals and a device for reproducing images. Therefore, some thumbnail s cannot be regenerated or generated depending on the synthesis, which is a problem.

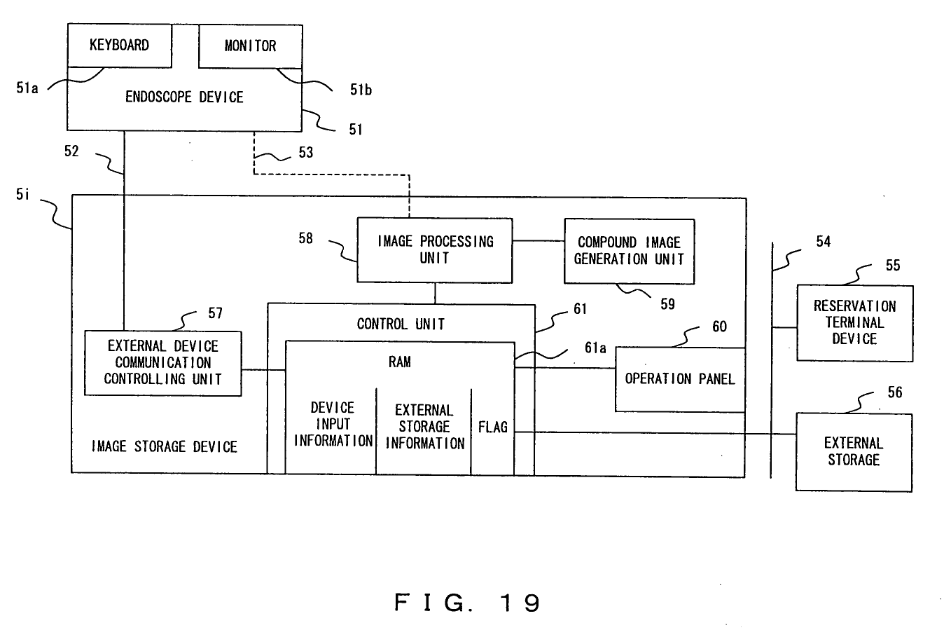

FIG. 17 shows one example of the system including the conventional image storage device.

FIG. 17 shows two systems. In the upper system of FIG. 17, an image storage device 46a is connected to an endoscope device 47a, and is also connected to an image regeneration device 48a, a server device 49a and the like via a network. In the lower system of FIG. 17 too, similarly, an image storage device 46b is connected to an endoscope device 47b, and is connected to an image regeneration device 48b, a service device 49b and the like via a network. The endoscope devices 47a and 47b are, for example, a device for outputting SDTV (standard definition television) type and HDTV (high definition television) type video signals, respectively.

In both the systems, a plurality of pieces of thumbnail data (thumbnail information) of thumbnail which become indexes for viewing images, based on video signals outputted from the endoscope device 47 is generated by the image storage device 46, and is stored in the server device 49 via the network. The plurality of pieces of thumbnail data is read and displayed on the image regeneration device 48 as requested. However, in the upper system of FIG. 17, the image storage device 46a generates thumbnail data of a small thumbnail size in accordance with a required small thumbnail size. However, in the lower system of FIG. 17, the image storage device 46b generates the thumbnail data of a large thumbnail size in accordance with a required large thumbnail size. Thus, the generated thumbnail size differs in both the systems. For example, the image storage device 46a cannot generate the thumbnail data of a large thumbnail size required by the lower system of FIG. 17.

As described above, conventionally, since the respective thumbnail sizes of the thumbnail data generated by the image storage device differ due to the difference between the systems, the same image storage device cannot be shared by different systems, which is a problem.

Therefore, in the image storage device of this preferred embodiment, the respective thumbnail data of a plurality of thumbnail sizes can be generated.

FIG. 18 shows one example of the system including the image storage device of this preferred embodiment.

FIG. 18 shows two systems. The upper system shown in FIG. 18 can be obtained by replacing the image storage device 46a in the upper system of FIG. 17 with an image storage device 5h of this preferred embodiment. The lower system shown in FIG. 18 can be obtained by replacing the image storage device 46b in the lower system of FIG. 17 with the image storage device 5h of this preferred embodiment. The server devices 49a and 49b are omitted in FIG. 18.

The image storage device 5h can generate a plurality of pieces of image data each with a different thumbnail size by selecting and setting a thumbnail size. For example, the upper system of FIG. 18 is set and used in such a way as to generate the thumbnail data of a small thumbnail size, and the lower system of FIG. 18 is set and used in such a way as to generate the thumbnail data of a large thumbnail size. The other configuration of the image storage device 5h is the same as the image storage device 5 of the first preferred embodiment.

As described above, according to this preferred embodiment, since the thumbnail data of a thumbnail with a size corresponding to its system can be generated by modifying the setting of the relevant device according to the system applied to the image storage device, the same image storage device can be applied to a plurality of systems. Since only one image storage device is needed for a plurality of systems, its maintenance can be simplified.

Although in the image storage device of this preferred embodiment, the thumbnail data of a corresponding thumbnail size by its setting, for example, device information (such as type information, etc.) can also be obtained from a connected device, and the thumbnail data of a corresponding thumbnail size can also be generates according to the device information. In this case, when the image storage device 5h obtains device information from the endoscope device 47a or image regeneration device 48a, in the upper system of FIG. 18, it generates the thumbnail data of a small thumbnail size. In the lower system of FIG. 18, when the image storage device 5h obtains device information from the endoscope device 47b or image regeneration device 48b, it generates the thumbnail data of a large thumbnail size.

### The Ninth Preferred Embodiment

Next, the ninth preferred embodiment of the present invention is described.

In a system including the conventional image storage device used in the endoscopic inspection department, already registered patient information which is read from a database separately from patient information inputted via an endoscope device exists. Therefore, even when patient information to be used is wrongly selected or is wrongly inputted, the process of the selection and the like is not recorded and as a result, there is a possibility that correct patient information may not be recorded. Since in the conventional system, priority is given to patient information already registered in a database even when the surname of a patient changes due to marriage or the like, the patient information cannot be modified by input via the endoscope device, which is a problem.

Therefore, in this preferred embodiment, a plurality of pieces of patient information can be correctly used, and correct patient information can be always registered in the database.

FIG. 19 shows one example of the system including the image storage device of this preferred embodiment.

As shown in FIG. 19, an image storage device 5i is connected to an endoscope device 51 via a video line 53 and a communication line 52. A reservation terminal device (inspection information pre-input device) 55, an external storage 56 which is a database (DB) and the like are also connected to the image storage device 5i via a network 54.

Although in this example, the endoscope device 51 is connected to the image storage device 5i, a plurality of devices, such as the supersonic wave endoscope device 2, external device 4 and the like can also be connected to the image storage device 5i as devices for outputting information to be obtained, as in the image storage device 5 of the first preferred embodiment.

The endoscope device 51 comprises a keyboard 51a for receiving a variety of inputs and a monitor 51b for displaying a variety of information and the like.

The image storage device 5i comprises an external device communication controlling unit 57, an image processing unit 58, a compound image generation unit 59, an operation panel 60 and a control unit 61. The external device communication controlling unit 57 controls communication with the endoscope device 51 via the communication line 52. The image processing unit 55 transmits/receives video signals to/from the endoscope device 51 via the video line 53, and performs a predetermined image process as requested. The compound image generation unit 59 generates a predetermined compound image as requested. The operation panel 60 receives a variety of inputs. The control means 61 controls the entire operation of the image storage device 5i.

The control unit 61 internally comprises RAM 61a. The RAM 61 comprises a device input information area, an external storage information area and a flag area. The device input information area stores patient information inputted via the keyboard 51a of the endoscope device 51. In the external storage information area, patient information stored (registered) in the external storage 56 is read and stored. In this example, the patient information includes at least one piece of information about a patient ID, a patient name, date of birth, sex, age and the like. The flag area stores a priority flag (priority flag indicating the priority of patient information) indicating to which priority is given, patient information stored in the device input information area or patient information stored in the external storage information area. The patient information to which priority is given by the priority flag is outputted to the endoscope device 51 via the communication line 52, and is displayed on the monitor 51b of the endoscope device 51. If this priority flag indicates that priority is given to patient information stored in the device input information area, the input and modification of the patient information via the endoscope device 51 is permitted. If it indicates that priority is given to patient information stored in the external storage information area, the input and modification of this patient information via the endoscope device 51 is prohibited.

Respective pieces of information stored in the device input information area, external storage information area and priority flag area are stored (registered) in the external storage 56 as requested. However, when it is stored, they are related with its image information and are stored with its image information.

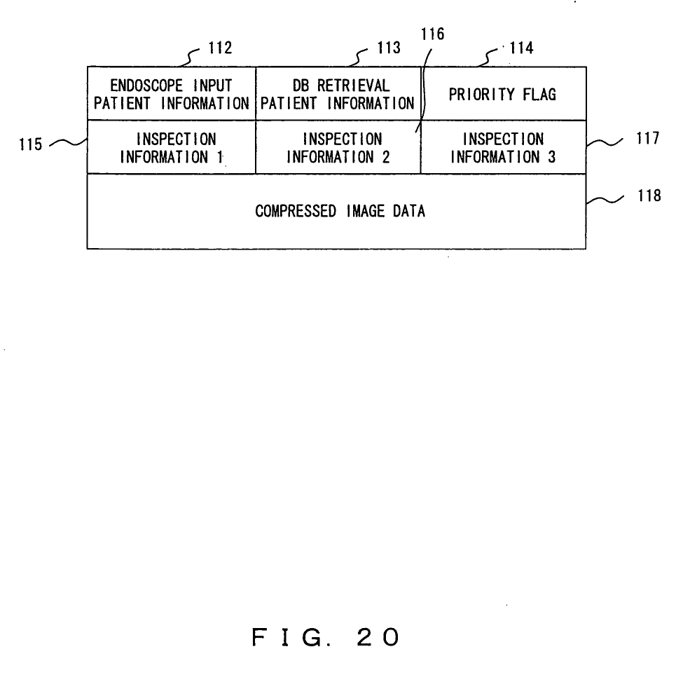

FIG. 20 shows one example of information stored in the external storage 56 at that time. In FIG. 20, a plurality of pieces of information stored in of the device input information area, external storage information area and priority flag area of RAM 61A are endoscope input patient information 112, DB retrieval patient information 113 and a priority flag 114, respectively, and are shown as inspection information 1 (115 in FIG. 20), inspection information 2 (116 in FIG. 20) and inspection information 3 (117 in FIG. 20), respectively. Compressed image data 118 indicates image information that is related and stored.

The reservation terminal device 55 connected to the image storage device 5i via the network 54 receives the input of patient information (inspection information) beforehand and so on. The external storage 56 stores (registers) the patient information received by the reservation terminal device 55, the information outputted from the image storage device 5i, which is shown in FIG. 20 and the like.

In a system with such a configuration, at the time of a normal endoscope inspection, a priority flag indicating that priority is given to patient information stored in the device input information area is set (stored) in the priority flag area of the RAM 61a, and the input and modification of the patient information via the endoscope device 51 is permitted. In this case, when the patient information is inputted via the endoscope device 51, it is stored in the device input information area of the RAM 61a. Simultaneously the patient information is outputted to the endoscope device 51 and is displayed on a monitor 51b. In this case, if patient information stored in the external storage 56 can be specified based on the inputted patient information, for example, when a patient ID or both of a patient name and date of birth is inputted as patient information, the external storage 56 is retrieved based on the inputted patient information. When corresponding patient information is retrieved, the patient information is read and stored in the external storage information area of the RAM 61a. If the patient information stored in the external storage information area includes information not included in the patient information in the device input information area, the information is outputted to the endoscope device 51 and is displayed on the monitor 51b. Thus, information other than one inputted via the endoscope device 51 is also displayed on the monitor 51b. In this case, if patient information is modified via the endoscope 51, patient information after the modification is stored in the device input information area of the RAM 61a and also is displayed on the monitor 51b of the endoscope device 51.

If patient information is inputted beforehand via the reservation terminal device 55 and is stored (registered) in the external storage 56, the patient information is stored in the external storage information area of the RAM 61a, and also a priority flag indicating that priority is given to patient information stored in the external storage information area is set (stored) in the priority flag area. In this case, the patient information stored in the external storage information area is outputted to the endoscope device 51 and is displayed on the monitor 51b, according to the set priority flag. However, in this case, since the input and modification of patient information via the endoscope device 51 is prohibited, according to the priority flag stored in the priority flag area, the patient information cannot be inputted nor modified. Thus, patient information is prevented from being modified, for example, by wrongly inputting it and so on when checking patient information registered beforehand.

The setting of a priority flag stored in this priority flag area can be freely modified by an instruction issued via the operation panel 60. Therefore, for example, by modifying the priority flag set when registering patient information in the external storage 56 via the reservation terminal device 55 beforehand to a priority flag indicating that priority is given to patient information stored in the device input information area, the patient information can be modified via the endoscope device 51.

As described above, according to this preferred embodiment, patient information can be correctly inputted by separately using patient information inputted via an endoscope device at the time of endoscopic inspection and patient information registered in the external storage 56 beforehand. Since as shown in FIG. 20, patient information inputted via an endoscope device and patient information read from the external storage 56 can be separately stored, an operating status and the like can be checked later. Even when there is an error in patient information, it can also be corrected without fail. By using a priority flag, priority can be given to an appropriate input method, thereby enabling the efficient and accurate input of patient information.

### The Tenth Preferred Embodiment

Next, the tenth preferred embodiment of the present invention is described.

There is a system including the conventional image storage device for obtaining image information from video signals outputted from an endoscope device or the like and registering (storing) it with its patient information in a database. In such a system, if image information is registered when patient information is not inputted or wrongly inputted, a temporarily patient ID is automatically generated and is registered after attaching it to the image information. However, as to image information registered in this way, it cannot be specified to which inspection device it belongs or to which inspection it belongs. If image information with the same temporary patient ID as one already registered in a database is registered, there is a possibility that the already registered image information may be overwritten by the patient information with the same temporary patient ID.

Therefore, in the image storage device of this preferred embodiment, image information can be recorded after attaching peculiar information such that it can be specified to which inspection device or to which inspection the relevant image information belongs as a temporary patient ID.

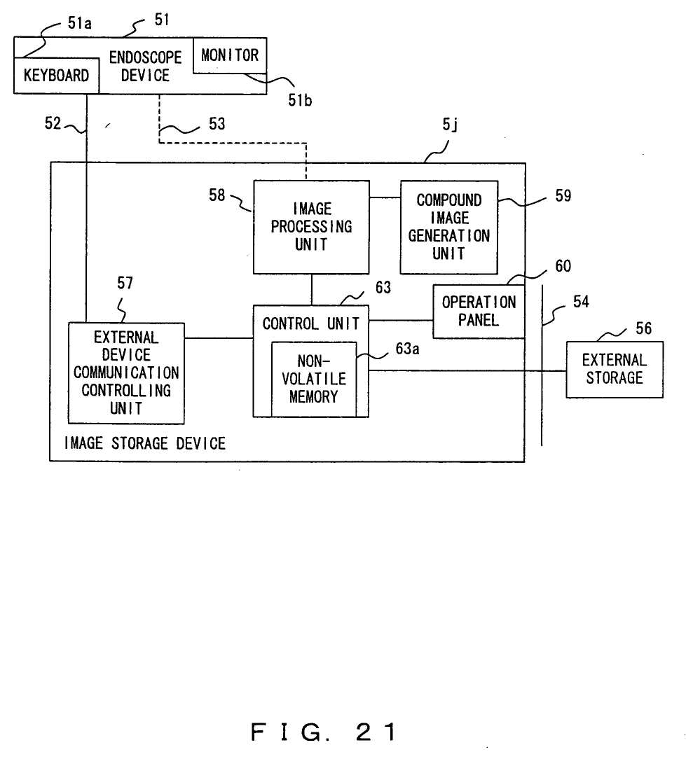

FIG. 21 shows one example of the system including the image storage device of this preferred embodiment. In FIG. 21, the same reference numerals are attached to the same components as in FIG. 19.

In FIG. 21, the control unit 63 of an image storage device 5j controls the entire operation of the image storage device 5j, and internally comprises non-volatile memory 63a. The non-volatile memory 63a stores the serial number (number peculiar to the device 5j) or MAC (Media Access Control) address used for network communication, of the image storage device 5j as the information of temporary patient ID. These serial number and MAC address are inputted via the operation panel 60. Alternatively, the MAC address can be automatically obtained. The other configuration is the same as that shown in FIG. 19. However, in FIG. 21 the reservation terminal device 55 shown in FIG. 19 is omitted.

Although in this example, the endoscope device 51 is connected to the image storage device 5j, a plurality of devices, such as the supersonic wave endoscope device 2, external device 4 and the like can also connected to the image storage device 5j as devices for outputting information to be obtained like the image storage device of the first preferred embodiment.

In a system with such a configuration, when an inspection is started, usually, a patient ID inputted via the keyboard 51a of the endoscope device 51 is attached to image information obtained from video signals from the endoscope device 51 via the video line 53 and is stored in the external storage 56 via the network 54.

However, if image information is stored in the external storage 56 without an patient ID since inspection is started without inputting patient information (including a patient ID) via the keyboard 51a of the endoscope device 51 or if patient information cannot be obtained due to the failure of the communication line 52, a patient ID cannot be obtained. In such a case, a temporary patient ID which includes a serial number or an MAC address recorded in the non-volatile memory 63a is automatically generated and is attached to image information, then, the image information is stored in the external storage 56.

The temporary patient ID generated automatically at this time, which includes the serial number or MAC address, consists of the combination among the serial number, MAC address, a release number, and the starting date of record. The temporary patient ID is selected from among six as follows by instruction done through the operation panel 60. That is, one is selected as the temporary patient ID from among (1) serial number+release number, (2) serial number+starting date of record, (3) serial number+release number+starting date of record, (4) MAC address+release number, (5) MAC address+staring date of record, and (6) MAC address+release number+starting date of record.

In this case, a release number begins from 0001 and is incremented every release. If a release number is used as the temporary patient ID, a temporary patient ID is generated and attached to each piece of image information for one image. If a release number is not used, the same temporary patient ID is generated and attached to a plurality of pieces of image information for consecutive images, which constitute one group. If a release number and the starting date of record are combined and used, the release number is reset at the starting time of one inspection day.

As described above, according to this preferred embodiment, if a patient ID cannot be obtained, either a serial number peculiar to an image storage device or an MAC address is attached to image information as the temporary patient ID. Therefore, a device that obtains image information can be easily specified. If a release number is used as the temporary patient ID, the recording order of a plurality of pieces of image information can be easily distinguished. The setting of a temporary patient ID attached to image information can also be modified in such a way that the image information can be easily managed for each device or for each inspection.

### The Eleventh Preferred Embodiment

Next, the eleventh preferred embodiment of the present invention is described.

In this preferred embodiment, if a plurality of devices, such as an endoscope device, a supersonic wave endoscope device and the like, are connected to an image storage device, an inspection starting time and an inspection end time which are needed to specify an inspection time can be obtained based on a control command outputted from one connected device.

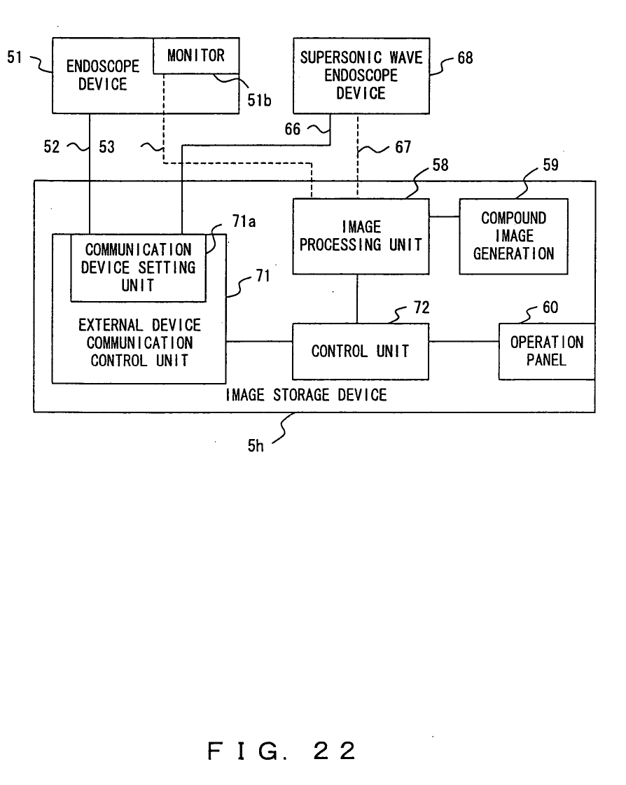

FIG. 22 shows one example of the system including the image storage device of this preferred embodiment. In FIG. 22, the same reference numerals are attached to the same components as in FIG. 19.

In FIG. 22, an image storage device 5h is connected to a supersonic wave endoscope device 68 via a video line 66 and a communication line 67 in addition to the endoscope device 51. An external device communication control unit 71 controls communication between the image storage device 5h and the endoscope device 51 via the communication line 52, and controls communication between the image storage device 5h and the supersonic wave endoscope device 68 via the communication line 66. A communication device setting unit 71a provided for the external device communication control unit 71 sets the correspondence between each of a plurality of communication ports provided for the image storage device 5h and each of devices connected to them and a communication port to which a device for receiving an inspection start command and an inspection end command with priority, of the plurality of connected devices is connected, according to the instruction done through the operation panel 60. Thus, an inspection start time and an inspection end time are obtained by the inspection start command and inspection end command, respectively, which are outputted from the device connected to the communication port set by the communication device setting unit 71a, thereby specifying an inspection time.

In this preferred embodiment, the image processing unit 58 also receives video signals from the supersonic wave endoscope device 68 via the video line 67. The control unit 72 controls the entire operation of the image storage device 7h.

The other configuration is the same as shown in FIG. 19. However, in FIG. 22, the network 54, reservation terminal device 55 and external storage 56 shown in FIG. 19 are omitted.

In a system with such a configuration, a setting by the communication device setting unit 71a is made as follows. Firstly, when an instruction to display the operation screen of the setting is issued via the operation panel 60, the operation screen is generated by the compound image generation unit 59, is outputted to the endoscope device 51 via the video line 53 and is displayed on the monitor 51b. Then, a setting is instructed according to the operation screen via the operation panel 60, the communication device setting unit 71a sets the correspondence between each of a plurality of communication ports provided for the image storage device 5h and each of devices connected to them and a communication port to which a device for receiving an inspection start command and an inspection end command with priority, of the plurality of connected devices is connected, according to the setting instruction.

In this case, it is assumed that the image storage device 5h comprises a plurality of communication ports including communication ports A and B, and that the correspondence between a communication port and a device, such as the endoscope device 51 as a device connected to the communication port A and the supersonic wave endoscope device 68 as a device connected to the communication port B, and the communication port A as a communication port to which a device for receiving an inspection start command and an inspection end command with priority, of the plurality of connected devices is connected, are set.

The reason why communication port A is set is as follows. Although in an endoscopic inspection, generally an endoscope device is used during the entire inspection period, the use of a supersonic wave endoscope device is limited to a part of the period and there is an unused period. Therefore, an inspection start time and an inspection end time are obtained by an inspection start command and an inspection end command which are outputted from an endoscope device, and an inspection time is specified.

After such a setting, an actual endoscopic inspection is started.

Firstly, before the inspection is started, the endoscope device 51 is activated and patient information is inputted via the endoscope device 51. Then, the inspection is started using the endoscope device 51, an inspection start command is issued from the endoscope device 51. In this case, since an inspection start time is obtained by the inspection start command from the endoscope device 51 by the above-mentioned setting of a communication port, a time the inspection start command is received is obtained as its inspection start time.

In this endoscopic inspection, the supersonic wave endoscope device 68 is used as requested. In this case, when the supersonic wave endoscope device 68 is activated and an inspection is started using the supersonic wave endoscope device 68, an inspection start command is received from the supersonic wave endoscope device 68. However, in this case, no inspection start time is obtained by this inspection start command due to the above-mentioned setting of a communication port. After the inspection by the supersonic wave device 68 is completed, an inspection end command is received from the supersonic wave endoscope device 68. However, in this case, no inspection end time is obtained by the inspection end command due to the above-mentioned setting of a communication port.

After the completion of the inspection by the endoscope device 51 is completed, an inspection end command is issued from the endoscope device 51. In this case, since it is set so that an inspection end time is obtained by the inspection end command from the endoscope device 51, as described above, a time the inspection end command is received is obtained as its inspection end time.

Then, the endoscopic inspection terminates.

By such a system operation, inspection start and end times are obtained by the inspection start and end commands outputted from the endoscope device 51, and the inspection time of the endoscopic inspection is specified. These inspection start and end times, inspection time and the like are recorded in the relevant image storage device 5h.

As described above, according to this preferred embodiment, even when a plurality of devices are connected to an endoscope device, an inspection time can be specified by a control command outputted from one device of them by setting. Therefore, for example, the inspection time of an endoscopic inspection can be accurately specified by setting so that the inspection time can be specified by a control command outputted from an endoscope device generally used during the entire endoscopic inspection period.

### The Twelfth Preferred Embodiment

Next, the twelfth preferred embodiment of the present invention is described.

In this preferred embodiment, when an image storage device recording image information, according to instructions, compressed image information obtained by compressing original image information can be recorded or the original image information (uncompressed image information) can be recorded without applying any process to it.

The original image information, which is uncompressed image information, is also based on DICOM, which is a standard for medical images and communication.

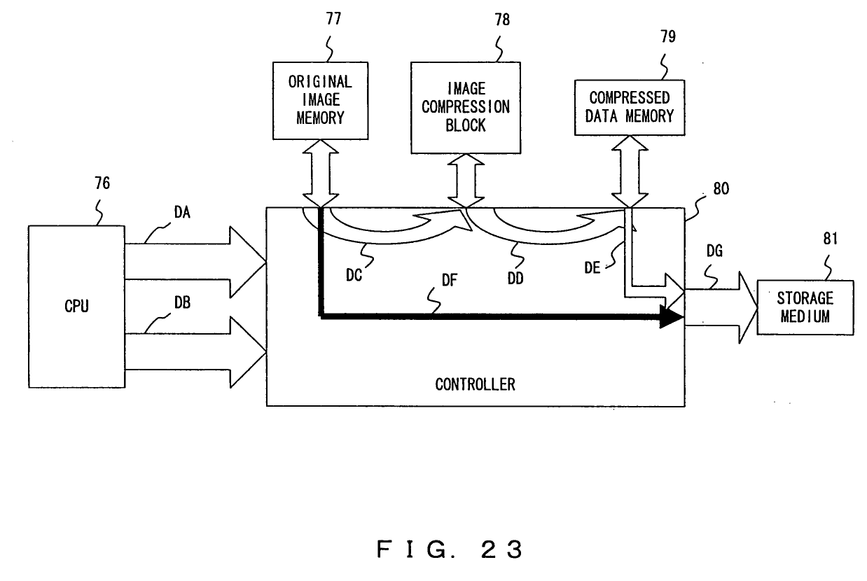

FIG. 23 shows one example of the system including the image storage device of this preferred embodiment.

In FIG. 23, the image storage device comprises a CPU 76 for controlling the operation of the entire relevant device, original image memory 77 for storing obtained original image information, an image compression block 78 for compressing the original image information, compressed data memory 79 for storing compressed original image information (compressed image information), a controller 80, which is an original image compression block controller and also a memory controller, a storage medium 81 for recording original image information or compressed image information and the like. The other configuration is the same as in the image storage device 5 of the first preferred embodiment.

The image storage device with such a configuration records compressed image information obtained by compressing the obtained original image information, or records the original image information without applying any process to it, according to instructions from a user.

If recording compressed image information obtained by compressing the obtained original image information, according to instructions from a user, firstly the CPU 76 issues an instruction to activate the image compression block 78 to the controller 80 (arrow mark DA). Thus, the image compression block 78 reads the original image information from the original image memory 77 storing the obtained original image information (arrow mark DC) and compresses the original image information and stores the compressed original image information (compressed image information) in the compressed data memory 79 (arrow mark DD). Then, the CPU 76 issues an instruction to transfer the compressed image information to the controller 80 (arrow mark DA). Thus, the compressed image information stored in the compressed data memory 79 is transferred to the storage medium 81 (arrow marks DE and DG).

If recording the obtained original image information without applying any process to it, according to instructions from a user, the CPU 76 issues an instruction to transfer the original image information to the controller 80 (arrow mark DB). Thus, the obtained original image information is read from the original image memory 77 to the storage medium 81 (arrow marks DF and DG). In this case, the original image information is transferred by one dot of a RGB array like RGBRGBRGB . . . Specifically, original image information is sequentially transferred by one dot of an RGB array from the left top corner of the image of the original image information to its right bottom corner.

As described above, according to this preferred embodiment, whether obtained original image information should be recorded without being compressed or it is compressed and recorded can be selected. As a result, for example, uncompressed image information, which is based on the DICOM standards can be outputted.

Although in this preferred embodiment, as described above, original image information is transferred by one dot of an RGB array when transferring it to the storage medium 81 (arrow marks DF and DG in FIG. 23), it can also be transferred for each plane of R, G and B. This transfer method (output method) for each plane of R, G and B is also based on DICOM, which is a standard for medical images and communication.

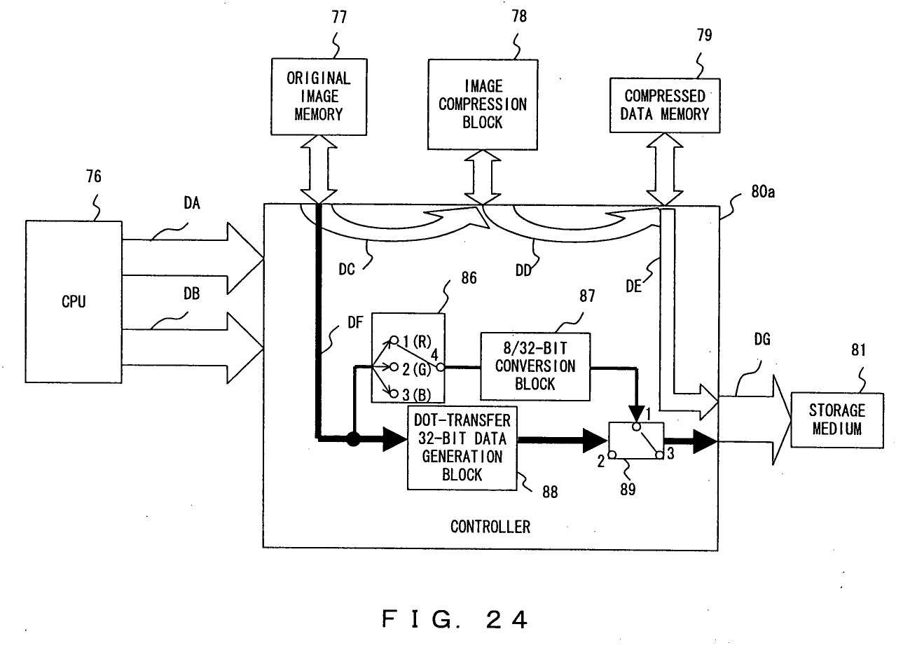

FIG. 24 shows an example of the configuration of an image storage device for transferring original image information for each plane of R, G and B. The image storage device shown in FIG. 24 differs from the image storage device shown in FIG. 23 only in the configuration of a controller, and the other configuration is the same as in it.

In FIG. 24, the controller 80a comprises two routes of a plane transfer route for transferring original image information for each plane of R, G and B and a dot transfer route for transferring it for each dot, as routes for transferring original image information stored in the original image memory 77, uncompressed. The plane transfer route comprises a selector 86 for switching a route according to the plane of R, G and B to be transferred and an 8/32-bit conversion block 87 for converting inputted 8-bit data into 32-bit data. In this example, it is assumed that each of R, G and B is expressed by 8 bits. The dot transfer route comprises a dot-transfer 32-bit data generation block 88 for generating 32-bit data to be transferred, based on inputted data. After the 8/32-bit conversion block 87 and the dot-transfer 32-bit data generation block 88, a selector 89 for switching a route according to an instruction about whether original image information should be transferred for each plane or for each dot is provided.

Since it is assumed that in the image storage device in this example, data is transmitted/received via a 32-bit bus, image information is converted to 32-bit data or 32-bit data is generated.

When transferring the obtained original image information to the storage medium 81, according to an instruction from a user, the image storage device with such a configuration transfers the original image information for each plane of R, G and B or for each dot.

If original image information is transferred for each plane of R, G and B, according to an instruction from a user, after the selector 89 is switched in such a way as to transfer the output of the 8/32-bit conversion block 87, the selector 86 is switched for each plane of R, G and B to be transferred. Then, the original image information is outputted for each plane of R, G and B via the 8/32-bit conversion block 87 and selector 89.

If original image information is transferred for each dot, according to an instruction from a user, after the selector 89 is switched in such a way as to transfer the output of the dot-transfer 32-bit data generation block 88. Then, the original image information is outputted for each dot via the dot-transfer 32-bit data generation block 88 and selector 89.

According to such a configuration, when transferring the obtained original image information uncompressed, whether the original image information should be for each plane of R, G and B or for each dot can be selected, and for example, the image information transfer method for each plane, which is based on the DICOM standards can be made compatible.

As described above, according to any of the image storage devices in the first through twelfth preferred embodiments of the present invention, if a variety of medical devices are complexly synthesized and used, a plurality of pieces of information provided by a plurality of devices can be obtained using one image storage device. Therefore, there is no need to provide a corresponding special device for each device, thereby realizing a favorable system capable of saving cost and space as a whole. Furthermore, since one image storage device can collectively handle a plurality of pieces of information provided by a variety if devices, information can be easily managed, which are advantages over the prior art.

### The Thirteenth Preferred Embodiment

Next, the thirteenth preferred embodiment of the present invention is described.

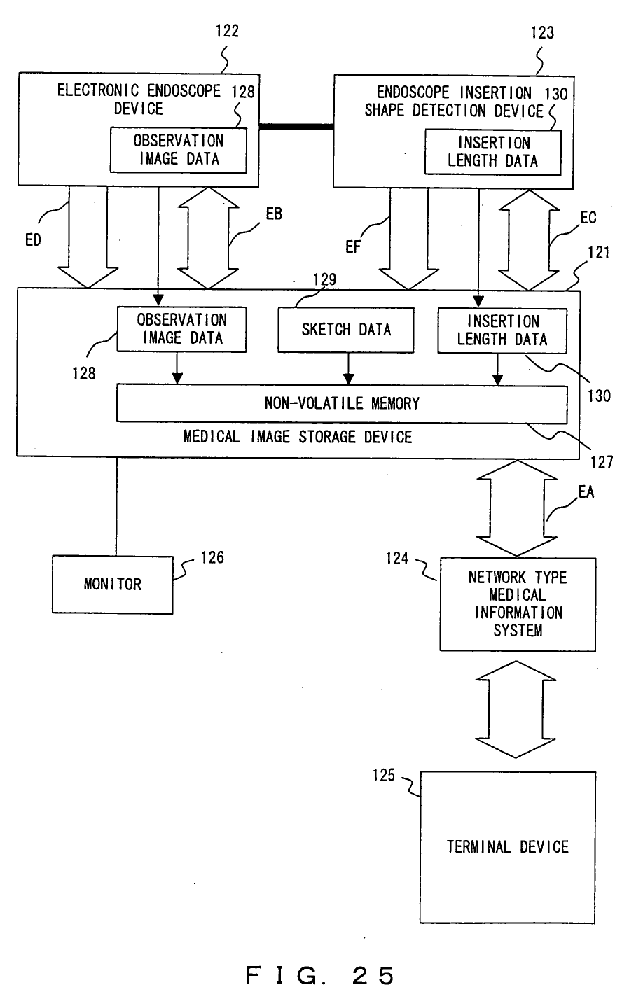

As shown in FIG. 25, this system comprises a medical image storage device 121, an electronic endoscope device 122, an endoscope insertion shape detection device 123, a network type medical information system 124, a terminal device 125 and the like. To the medical image storage device 121, a monitor 126 is connected.

For the electronic endoscope device 122 and endoscope insertion shape detection device 123 which constitute this system, widely used conventional medical devices are used. The endoscope insertion shape detection device 123 is connected to the electronic endoscope device 122 by a universal cord extended from a scope (endoscope).

The electronic endoscope device 122 and endoscope insertion shape detection device 123 are connected to the medical image storage device 121. Furthermore, the medical image storage device 121 conducts TCP/IP communication with the network type medical information system 124 (arrow mark EA). To the network type medical information system 124, the terminal device 125 for reading electronic cartes and inputting inspection reports is connected to a server, which is omitted in FIG. 25).

Between the medical image storage device 121 and the electronic endoscope device 122/endoscope insertion shape detection device 123, serial communication signals are transmitted/received by a RS-232C (arrow marks EB and EC), and the insertion length data of an endoscope, the release signal of en electronic endoscope device, equipment data and the like are transmitted. Between the electronic scope device 122 and the medical image storage device 121, signals of X contacts can also transmitted/received (arrow mark EB). The electronic endoscope device 122 and endoscope insertion shape detection device 123 transmit RGB video signals to the medical image storage device 121 (arrow marks ED and EF), and transmits the observation image data 128 of the electronic endoscope device 122.

As shown in FIG. 25, the medical image storage device 121 stores sketches (sketch data) 129 in predetermined memory, such as non-volatile memory (endoscope observation image storage unit/endoscope insertion length storage unit) 127 or the like beforehand. A sketch is electronic data representing the outline of the stomach, esophagus, large intestine or the like, and indicates the shape of a body part to be inspected by the electronic endoscope device 122.

As described above, since the endoscope insertion shape detection device 123 is connected to the electronic endoscope device 122, it can be detected length from a marker which indicates display beginning point of the endoscope to the endoscope, that is, insertion length of the endoscope. How many centimeters away from the entrance of an inspected body part an endoscope is located at a specific time if the insertion length of the endoscope is known. This is because the average size of each organ can be known. Since the electronic endoscope device 122, endoscope insertion shape detection device 123 and medical image storage device 121 are connected to each other, the observation image data of a morbid part, shot by the electronic endoscope device 122 can be linked with a sketch on the monitor 126 of the medical image storage device 121 and be promptly displayed on the monitor 126, based on the insertion length data 130 of the endoscope insertion shape detection device 123, when the morbid part is discovered.

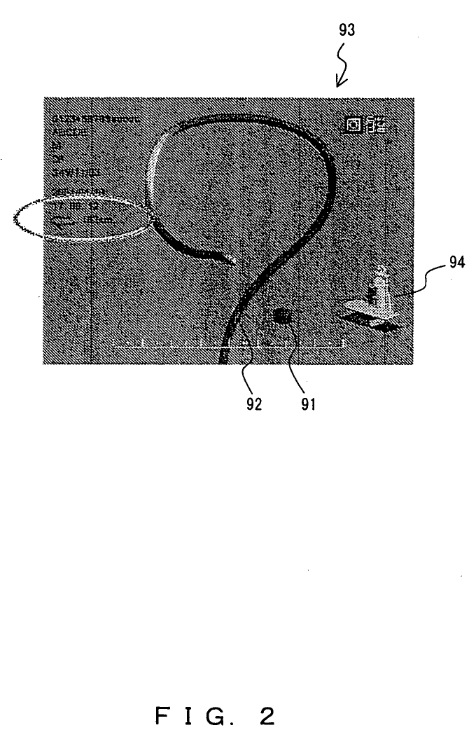

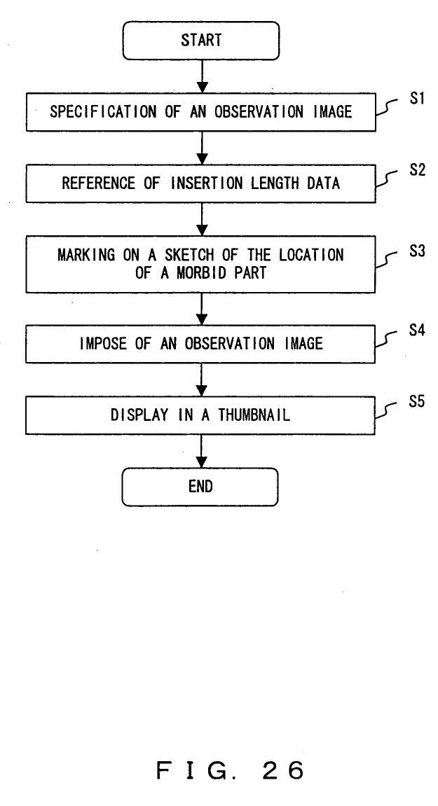

The process of taking the observation image shot by an endoscope into a sketch, using insertion length data is described below in detail. A flowchart showing the process of linking a sketch and the observation image of an endoscope is shown in FIG. 26. Each of parenthesized S1, S2, and so on in the following description indicates each step shown in FIG. 2.

It is presumed that a body part to be inspected is specified and a sketch is displayed on the monitor 126 of the medical image storage device 121. When an endoscopic inspection is started, the endoscope insertion shape data and insertion length data of the electronic endoscope device 122 is sequentially transmitted from the endoscope insertion shape detection device 123 to the medical image storage device 121. From the electronic endoscope device 122, observation image data shot by an endoscope connected to the electronic endoscope device 122 is sequentially transmitted to the medical image storage device 121. The medical image storage device 121 records the observation image data and insertion length data transmitted from time to time from the electronic endoscope device 122 and endoscope insertion shape detection device 123 in predetermined memory, such as non-volatile memory (endoscope observation image storage unit/endoscope insertion length storage unit) or the like, and monitors/manages it.

When discovering a morbid part in the observation image of an endoscope, the user of the medical image storage device 121 specifies an observation image in which the morbid part is shot by a pointing device or the like (“specification of an observation image”) (S1). Upon receipt of an instruction to specify an observation image, the medical image storage device 121 writes the observation image in memory. Then, the insertion length data received from the endoscope insertion shape detection device 123 is specified based on the time stamp of the written observation data (“reference of insertion length data”)(S2). How far away from the entrance of a body part to be inspected the morbid is located is calculated based on the read insertion length data, and the corresponding point is marked on the sketch (“marking on a sketch of the location of a morbid part”)(S3).

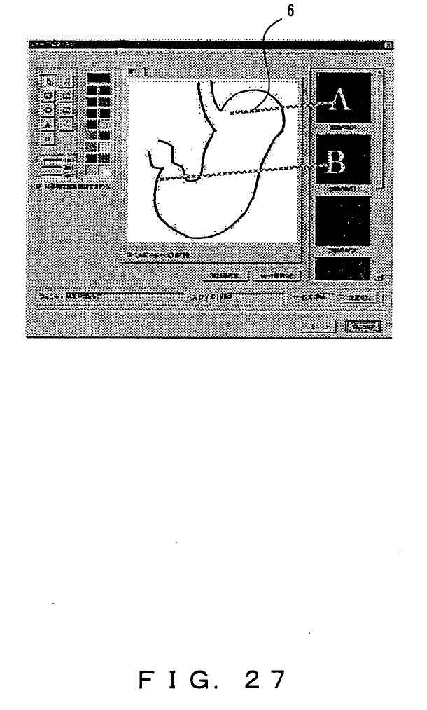

Then, the medical image storage device 121 reads the specified observation image data from the memory, reduced the observation image data so as to be accommodated out the frame of the screen displaying a sketch and imposes the observation image shot by the electronic endoscope device 122 on the screen displaying the sketch like the example of the sketch of a report linked and indicated shown in FIG. 27 (“impose of an observation image”) (S4). When the specified observation image data is imposed on the screen of the monitor 126, the marked point of the sketch and the observation image reduced and displayed are connected by a leader shown in FIG. 27 so that the user can link and read them. The imposed image is displayed in a thumbnail (“display in a thumbnail”) (S5). When the user clicks a thumbnail, the observation image with a normal size can be displayed on the monitor 126. The observation image data received from the electronic endoscope device 122 can be inserted in JPEG format or the like, which varies with a hospital, regardless of the storage method of a file.

As described above, according to this preferred embodiment, there is no need to specify the location of a morbid part on a sketch every time a morbid part is discovered, and it is automatically inputted. Accordingly, the operation can be simplified. Since the location of a morbid part can be specified without relying on the memory of an endoscopic inspector, all of morbid parts can be recorded without fail even when there are lots of morbid parts.

Alternatively, an image indicating the insertion shape of an endoscope and an endoscopic observation image can be simultaneously displayed. The user of the medical image storage device 121 can freely select whether an endoscope insertion shape and an observation image should be displayed simultaneously or separately.

Although in this application, apiece of thumbnail and the mark on a sketch are directly linked, sometimes lots of images are shot for one body part, depending on the type of a disease. In such a case, a list of thumbnails can also be displayed for each body part and one of them can also be selected as a thumbnail to be linked.

### The Fourteenth Preferred Embodiment

Next, the fourteenth preferred embodiment of the present invention is described.

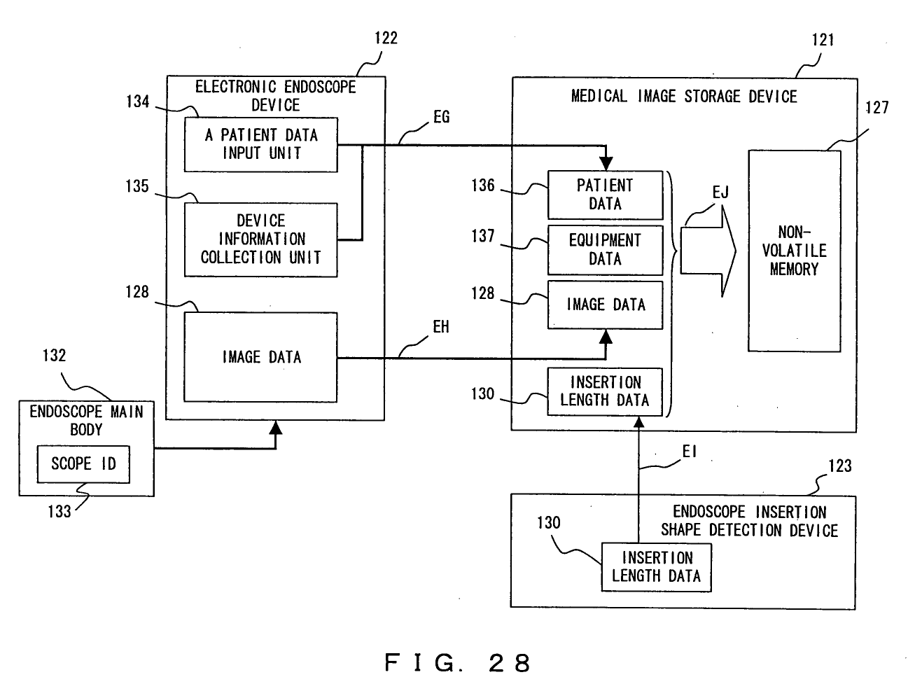

The basic configuration of a medical information system provided with the medical image storage device of the fourteenth preferred embodiment is the same as one shown in FIG. 25. Specifically, the medical information system comprises the medical image storage device 121, electronic endoscope device 122 and endoscope insertion shape detection device 123. However, in the fourteenth preferred embodiment, as shown in FIG. 28, a scope ID 133, which is data for identifying a scope (endoscope) used for an inspection, is managed within the electronic endoscope device 122 as the data of an endoscope main body 132.

Since the medical image storage device 121 is connected to the electronic endoscope device 122, the scope ID data can be also shared by the medical image storage device 121.

The medical image storage device 121 links each scope ID with the type of a scope, such as a general scope for upper digestive organs, a scope for the large intestine or the like, that is, a body part to be inspected and manages it. Therefore, when receiving scope ID data, the medical image storage device 121 can specify the body part to be inspected, based on the scope ID value.

Since the medical image storage device 121 stores the sketch of each body part to be inspected beforehand, the medical image storage device 121 can automatically display the sketch of the inspection body part specified by the scope ID data on the monitor 126 shown in FIG. 25 as the sketch of an organ to be inspected. Accordingly, there is no need for a user to specify and display the sketch of a body part to be inspected.

Furthermore, since the medical image storage device 121 records the insertion length of an endoscope together with a patient ID, an inspection report generated in this preferred embodiment can also be used for the process observation of a morbid part. For example, when a polyp is cut and removed from a point, such a report can be used for the re-inspection half a year later. In this case, since the point from which the polyp is cut and removed can be roughly predicted based on the insertion length data of the endoscope, a time needed to re-inspect it can be reduced. This is also effective when a plurality of doctors examine one patient.

Alternatively, an image indicating the insertion shape of an endoscope and an endoscopic observation image can also be simultaneously displayed on the monitor of the medical image storage device 121. In this case, the user of the medical image storage device 121 can freely select whether the endoscope insertion shape and observation image should be displayed simultaneously or separately.

In FIG. 28, the elements 134 and 135 of the electronic endoscope device 122 indicate a patient data input unit, such as a keyboard or the like, and a device information collection unit for collecting device information, such as a S/N, a scope type, the number in use of a scope and the like. Then, data about a release, patient data, equipment data and the like obtained by these unit 134 and 135 are transmitted to the medical image storage device 121 as communication signals (arrow mark EG). The image data 128 is transmitted from the electronic endoscope device 122 to the medical image storage device 121 as video signals (arrow mark EH). The insertion length data 130 of the endoscope insertion shape detection device 123 is also transmitted from the endoscope insertion shape detection device 123 to the medical image storage device 121 as communication signals (arrow mark EI). Then, the patient data 136, equipment data 137, image data 128, insertion length data 130 and the like transmitted to the medical image storage device 121 in this way are stored in the non-volatile memory 127 (arrow mark EJ).

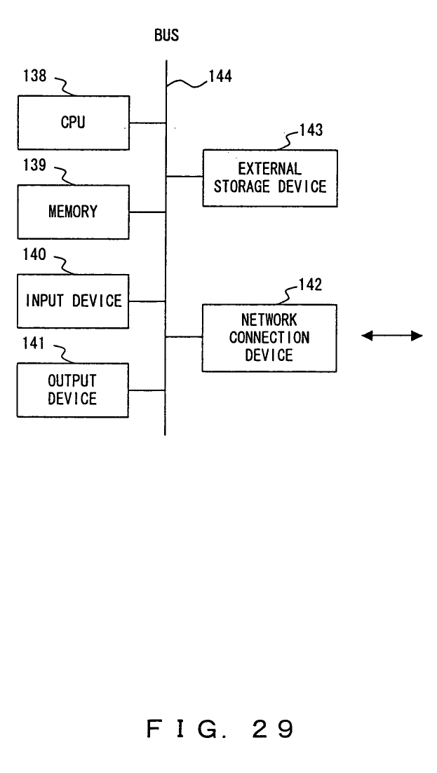

However, of the functions of the devices constituting this preferred embodiment, particularly, that of a data processing part is performed using an information processing device (computer, etc.) as shown in FIG. 29. The information processing device shown in FIG. 29 comprises a central processing unit (CPU) 138, memory 139, an input device 140, an output device 141, a network connection device 142 and an external storage device 143, which are all connected to each other by a bus 144.

The memory 139 includes read-only memory (ROM), random-access memory (RAM) and the like, and stores a program and data used for the process. The external storage device 143 also stores observation image data and the like. The CPU 138 links a series of observation images with a sketch and displays them, by using the memory 139 and running the program.

The input device 140 is used for a user to specify an observation image to be linked. The output device 141 displays an observation image linked with the sketch and a thumbnail.

The network connection device 142 connects the medical information system to an external medical device through a network. The medical image storage device 121 receives observation image data and the insertion length data of an endoscope from the electronic endoscope device 122 and endoscope insertion shape detection device 123, respectively, or from an external device via the network connection device 142, and loads them onto the memory 139 to use them.

Since the medical image storage device of this preferred embodiment comprises a means for linking observation image data with the sketch of a body part to be inspected and displaying the observation image data, it can also be used to inspect a body part without using an endoscope insertion shape detection device.

As described above, according to each of the thirteenth and fourteenth preferred embodiments of the present invention, a variety of data can be comprehensively utilized by managing data possessed by each medical device by a medical image storage device. Since it comprises a means for automatically linking the observation image of a morbid part of a body part to be inspected, of an endoscope with the morbid part on a sketch, a time needed to make a report can also be greatly shortened. Since the report is electronically written, it can also be used for purposes other than recording an inspection result.

### The Fifteenth Preferred Embodiment

Next, the fifteenth preferred embodiment of the present invention is described.

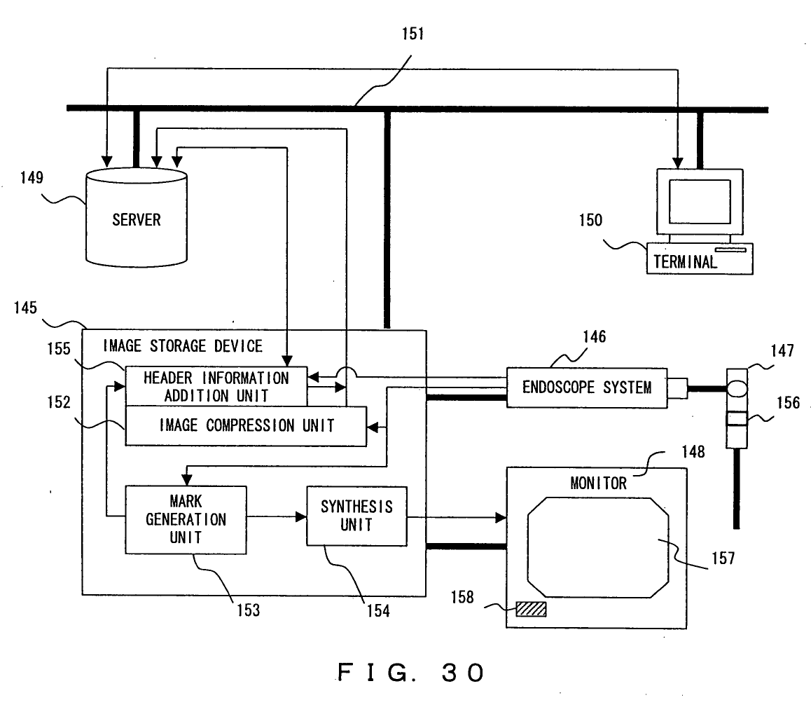

FIG. 30 shows the configuration of the endoscopic image filing system in this preferred embodiment.

The system shown in FIG. 30 comprises a marking function to attach a mark indicating its selection to an image shot while inspecting at the time of endoscopic inspection. Therefore, labor for looking for an image used to make a report can be reduced by marking an image used for a report while shooting it at the time of endoscopic inspection and making a report later selecting it from others.

The endoscopic image filing system shown in FIG. 30 comprises an image storage device 145, an endoscope system 146, a scope 147, a monitor 148, a server (server device) 149 and a terminal 150.

Of these components, the image storage device 145, server 149 and terminal are connected to a LAN 151. Images shot by the endoscope system 146 are recorded in the server 149 by the image storage device 145 via this LAN 151. When making a report, data, such as the images and the like stored in the server 149 is read by the terminal 150. To the image storage device 145, the endoscope system 146 and monitor 148 are connected via a dedicated line. This system can also comprise a plurality of the image storage devices 145 and/or terminals 150. Alternatively, one image storage device 145 can comprise a plurality of endoscope systems and/or scopes 147.

The image storage device 145 records/stores images taken in by an endoscope in the server 149 and displays shot images, a variety of information about the status of each connected device and the like on the monitor 148, for an operator, such as an endoscopic inspector and the like. The image storage device 145 comprises an image compression unit 152, a mark generation unit 153, a synthesis unit 154 and a header information addition unit 155.

The image compression unit 152 compresses image data from the endoscope system 146. The mark generation unit 153 generates image data with a selection mark when an operator, such as an endoscopic inspector or the like, operates a switch 156. The synthesis unit 154 synthesizes an endoscopic image inputted from the endoscope system 146 and a selection mark generated on a shot image by the mark generation unit 153 into display screen data and outputs it to the monitor 148. The header information addition unit 155 generates header information including patient information attached to compressed data and information about a shooting environment, when the unit 155 records the taken still image in the server 149. The image storage device 145 transmits image data to the server 149 as an image file in which header information is attached to the compressed data of the taken image and registers the endoscopic image in the database of the server 149.

The endoscope system 146 converts the observation image of an inspection object shot by the scope 147 into video signals and outputs it to the image storage device 145 as an endoscopic image.

The scope 147 shoots images. Its tip is inserted into a patient to be inspected and the reflection of light emitted by a light source, which is not shown in FIG. 30, is converted into electrical signals by a solid-state image sensing device, such as a charge-coupled device (CCD) or the like, which is outputted to the endoscope system 146. The scope 147 also comprises a scope switch 156 in a place where an operator can operate it while inspecting. For this scope switch 156, three or four button switches are usually provided, and a function to be set in each switch can be assigned by software by modifying its setting. In this example, as the scope switch 146, four button switches of SW_A, SW_B, SW_C and SW_D are provided. It is assumed that of these switches, SW_A, SW_B and SW_C are a release switch for taking in a shot image as still image data, a marking switch for setting the system in a marking mode for marking an image to be taken in as a still image and a start/end switch for notifying the start and end of an inspection, respectively. This function as a start/end switch can also be assigned to an input device, such as the keyboard, a foot switch or the like of the endoscope system 146, instead of being assigned to the scope switch.

The monitor 148 is a display monitor for displaying an endoscopic image 157 being currently shot by the scope 147. On the display screen of this monitor 147, a selection mark 158 indicating whether the system is currently in the marking mode is also displayed besides the endoscopic image 157. If an endoscopic image is taken in by pushing the release switch SW_A while this selection mark 158 is displayed on the monitor 148, the taken image is marked. Although it is described above that after the system enters into the marking mode by pushing the SW_B of the scope switch 156, a marked image is taken in by pushing SW_A, this preferred embodiment is not limited to this. Alternatively, an image can also be marked and taken in by pushing only SW_B.

The server 149 is a file server for making a database of the patient's personal endoscopic information, a shot image and the like and storing it. The terminal 150 is an information processing device connected to the server 149 via a network by LAN. An endoscopic inspector makes a report while calling up the endoscopic image and patient information taken in from the server 149, using this terminal 150.

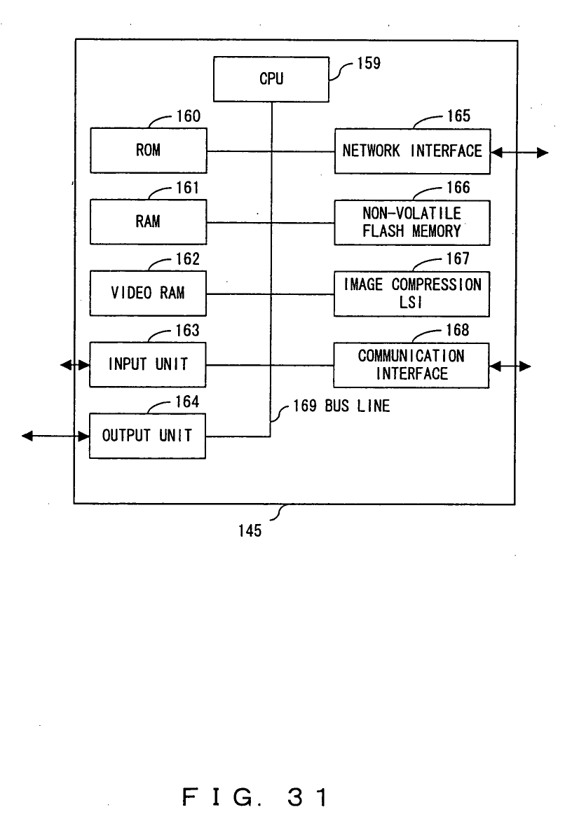

FIG. 31 shows an example of the configuration of the image storage device 145.

The image storage device 145 shown in FIG. 31 comprises a CPU 159 for performing a variety of operational control, ROM 160 for storing a control program and the like, RAM 161 as working memory at the time of operation, video RAM (V-RAM) 162 for temporarily storing video signals from the endoscope system 146, an input unit 163 for receiving an input from an input device, such as an operation panel, a keyboard or the like, and an input from a portable storage medium reading device, such as CD-ROM or the like, an output unit 164 for outputting image data and the like to a monitor 148, a network interface (network I/F) 165 for transmitting/receiving signals, such as data, instructions and the like to/from a network, non-volatile flash memory 166 for storing compressed image data to be transmitted to a server 149, an image compression LSI 167 for compressing video signals and a communication interface (communication I/F) 168 for transmitting/receiving communication signals to/from the endoscope system 146, which are all connected to each other by a bus line 169.

In the image storage device 145 shown in FIG. 31, the CPU 159 controls the other components by executing firmware in the ROM 160 and a program read from the input unit 163, and realizes a variety of processes set forth in this specification, such as a process for displaying data on the monitor 148, the marking process of endoscopic images, a process for storing data in the server 149 and the like. The image storage device 145 does not realize the process by software by the CPU 159 running a program as shown in FIG. 31. Instead, a part of the device 145 or the entire device 145 can also be configured by hardware and the process can also be realized.

Next, the operation at the time of endoscopic inspection of the system shown in FIG. 30 is described.

When conducting an endoscopic inspection, firstly, an operator, such as an endoscopic inspector or the like, inputs patient identification information such as an inspection ID attached to this endoscopic inspection and the ID number of a patient to be inspected and the like (if a patient is not registered in the server 149 yet, patient personal information, such as a name, age, sex, etc., are also inputted), using the input device, such as a keyboard, a card-reader or the like of the endoscope system 146, which is provided beside patient's bed. When they are inputted, the header information addition unit 155 of the image storage device 145 reads the personal information of this patient from the server 149 to generate header information to be attached to the image data of a taken endoscopic image.

Then, the operator notifies the endoscope system 146 of the start of the inspection by pushing the SW_C of the scope switch 156. By this notification, the header information addition unit 155 of the image storage device 145 records an inspection start time. This time is recorded based on the internal clock of the server 149.

The operator operates the scope 147 while watching an endoscopic image 157 displayed on the monitor 148, and takes in an endoscopic image as a still image by pushing the SW_A of the scope switch 156, which is a release button, if necessary. When SW_A is pushed, in the image storage device 145, the image compression unit 152 compresses image data inputted from the endoscope system 146, the header information addition unit 155 attaches header information composed of the personal information of the patient and the shooting information of the image and the like to this compressed image data. The compressed image data is transmitted to the server 149 via the LAN 151 and is stored in it.

Although the operator continues to take in an endoscopic image by pushing SW_A while operating the scope 147, the operator pushes SW_B before pushing release button SW_A if the operator takes in an image which may be used to make a report later. This pushing of SW_B is notified to the mark generation unit 153 via the endoscope system 146. By this notification, the mark generation unit 153 generates the image data of a selection mark to be displayed on the monitor 148 and outputs it to the synthesis unit 154. In this case, the mark generation unit 153 also notifies the header information addition unit 155 of the generation of the mark. Upon receipt of the image data, the synthesis unit 154 synthesizes the image data from the mark generation unit 153 and an endoscopic image from the endoscope system 146, and outputs it to the monitor 148. By his synthesis process, a selection mark 158 indicating its marking is displayed on the screen of the monitor 148. Thus, the operator can confirm that the image is selected while operating it.

If the operator takes in an endoscopic image by pushing SW_A while this selection mark 158 is displayed, as described above, the header information generated by the header information addition unit 155 is attached to the image data compressed by the image compression unit 152, which is transmitted to the server 149. In this case, upon receipt of this notice from the mark generation unit 153, the header information addition unit 155 switches a flag indicating the image in the header information is marked on. Thus, information indicating that the image is marked is attached to the endoscopic image data stored in the server 149.

If SW_B is pushed again before pushing SW_A after pushing SW_B, marking is cancelled, and the cancellation is notified to the mark generation unit 153 and header information addition unit 155. Thus, the mark generation unit 153 suspends the generation of a marked image, and the selection mark 158 indicating that marking is performed disappears from the display screen. Then, the header information addition unit 155 switches a flag indicating that an image is marked in the header information off. If SW_A is not pushed even when a specific time elapses after pushing SW_B, marking is cancelled and the selection mark 158 disappears from the display screen of the monitor 148.

After all the inspection is completed, the operator notifies the endoscope system 146 of the end of the inspection by pushing scope switch SW_C. By this notification, the header information addition unit 155 of the image storage device 145 records an inspection end time based on the internal clock of the server 149. Then, the header information addition unit 155 notifies the server 149 of this inspection end time and the above-mentioned inspection start time.

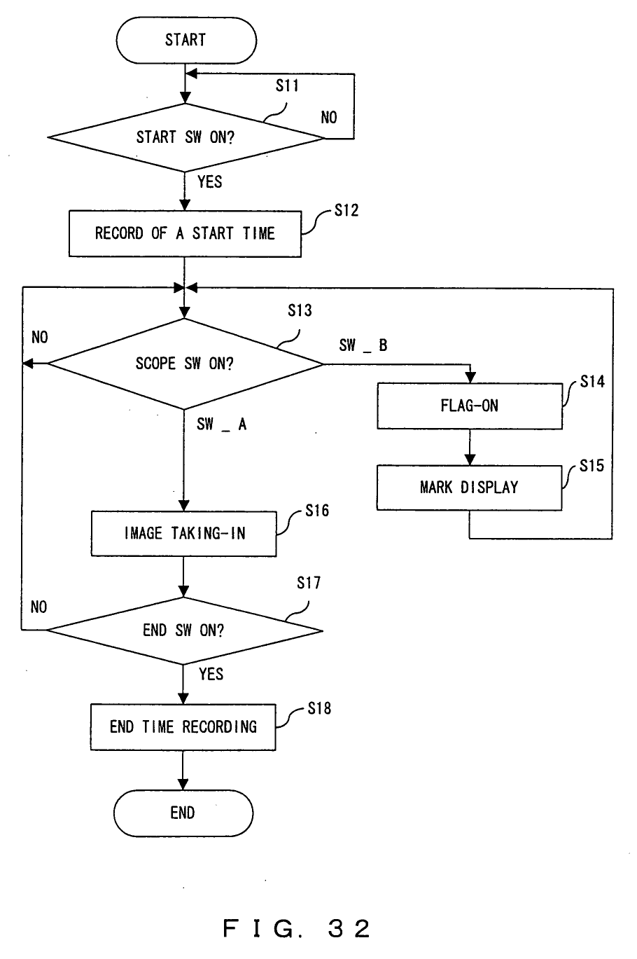

FIG. 32 is a flowchart showing the operational process of the image storage device 145 at the time of endoscopic inspection.

When an endoscopic inspection is started, firstly in step S11, the image storage device 145 waits for the SW_C, which is a start switch, being pushed (“start SW on?”) (NO in step S11). If SW_C is pushed (YES in step S11), in step S12, the server 149 is inquired of the current time and the answered time is recorded as an inspection start time (“record of a start time”). Then, in step S13, input of the scope switch 156 is awaited (“scope SW on?”)(NO in step S13).

If in step S13, the scope switch 156 is pushed and it is marking switch SW_B (SW_B in step S13), in step S14, the header information addition unit 155 switches a flag for indicating whether it is marked on, which is included header information added to compressed image data (“flag-on”). Then, in step S15, the mark generation unit 153 generates a mark image and displays a selection mark 158 on the monitor 148 (“mark display”). Then, the process returns to a switch input waiting status in step S13.

If in step S13, the scope switch 156 is pushed and it is release switch SW_A (SW_A in step S13), in step S16, an image shot by the scope 147 is taken in as a still image (“image taking-in”).

After that, the processes in steps 13 through 17 are repeated (No instep S17) until instep S17 end switch SW_C is pushed (“end SW on?”). If in step S17, SW_C is pushed (YES in step S17), in step S18, the server 149 is inquired of the current time and the answered time is recorded as an inspection end time (“end time recording”). Then, the process terminates.

After the endoscopic inspection is completed, an endoscopic inspector taking charge of the endoscopic inspection makes an inspection report for reporting the result of the endoscopic inspection to use it as a report to a doctor in charge, an electronic carte or so on.

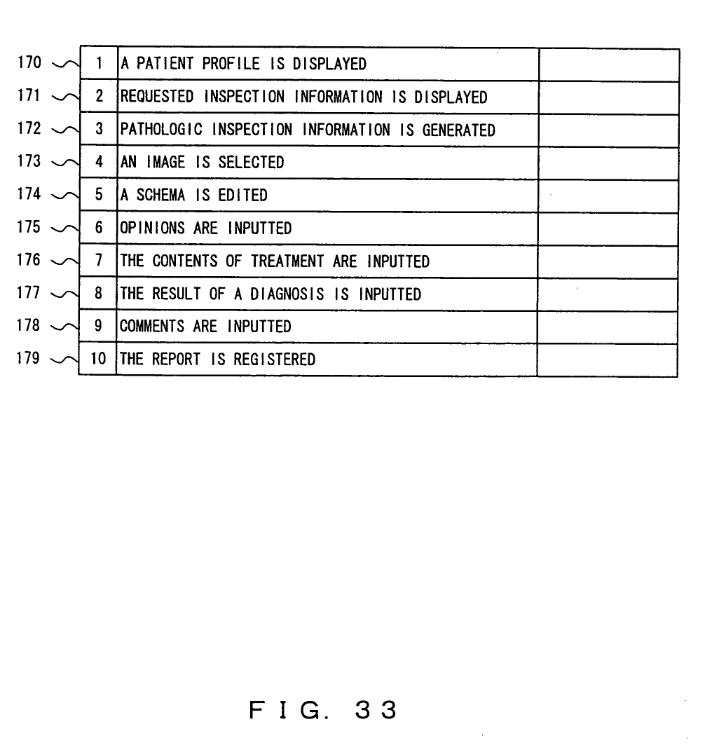

FIG. 33 shows an example of the procedure of issuing a report.

An inspection report is issued while the terminal 150 is reading data stored in the server 149. In this example, the report is issued in the following procedure. Firstly, a patient profile is displayed (170 in FIG. 33). In this case, if it is requested by another medical department, requested inspection information is displayed (171 in FIG. 33). Then, pathologic inspection information is generated (172 in FIG. 33) and an image is selected (if an image is selected here, etc.)(173 in FIG. 33). Then, a schema for inputting the sketch of an inspected body part is edited (174 in FIG. 33) and opinions are inputted (175 in FIG. 33). Then, the contents of treatment given during inspection are inputted (176 in FIG. 33) and the result of a diagnosis is inputted (177 in FIG. 33). Lastly, comments are inputted (178 in FIG. 33). After the report is completed, the report is registered (179 in FIG. 33), specifically, the completed report is registered in the database of the server 149.

In procedure 4 image selection (173 in FIG. 33) of the report issuance procedure shown in FIG. 33, conventionally, after images shot at the time of inspection are taken in the terminal 150, all the images are displayed and so on, their contents are sequentially checked depending on memory and images to be used for a report are selected.

However, in the system of this preferred embodiment, since endoscopic images to be used for a report, such as images in which a polyp is detected, are marked when taking in them at the time of endoscopic inspection, the image to be used can be easily selected by checking the marked images.

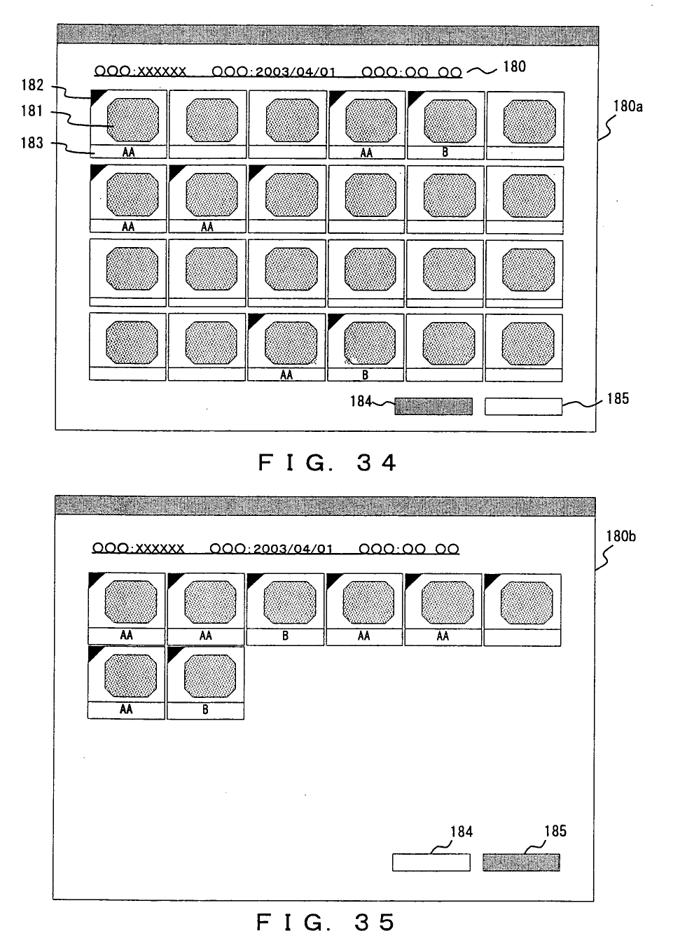

FIGS. 34 and 35 show examples of the image selection screen used in the image selection of the report issuance procedure.

When operating and selecting a tab for a list of images or pull-down menu on the report issuance display screen of the terminal 150, as shown in FIG. 34 as the image selection screen, a list of the thumbnails of endoscopic images taken in as index images is displayed on the screen of the terminal 150.

In FIG. 34, a list of the thumbnails 181 of all the endoscopic imaged shot during the inspection is displayed below a variety of information 180 about an endoscopic inspection whose report is issued, such as its inspection ID, the date of the inspection, the name of a patient and the like.

A selection mark 182 is attached to each of images marked when taking in them, of the thumbnails 181 displayed on the screen, therefore, a report maker can easily detect images that are selected for a report and marked on the display screen 180a, and images necessary for a report can be easily detected by mainly checking these marked images.

The report maker can enlarge an arbitrary thumbnail 181 by selecting it using a pointing device, such as a mouse or the like. If the report maker selects a button (marked image button) 185 by the pointing device on the display screen 180a, only marked images can be selected and displayed as shown in FIG. 35. If he/she selects a button (all image button) 184 by the pointing device on the display screen 180b, the display screen can be returned to the display screen 180a for displaying all images.

The report maker continues to select images by checking images taken in during inspection and operating a tab 183, which is a pull-down menu, by the pointing device, on the display screen 180 shown in FIG. 34 or 35. In FIGS. 34 or 35, five images (whose tab 183 is described as AA) and two images (whose tab 183 is described as B) are selected as ones for the esophagus and the stomach, respectively.

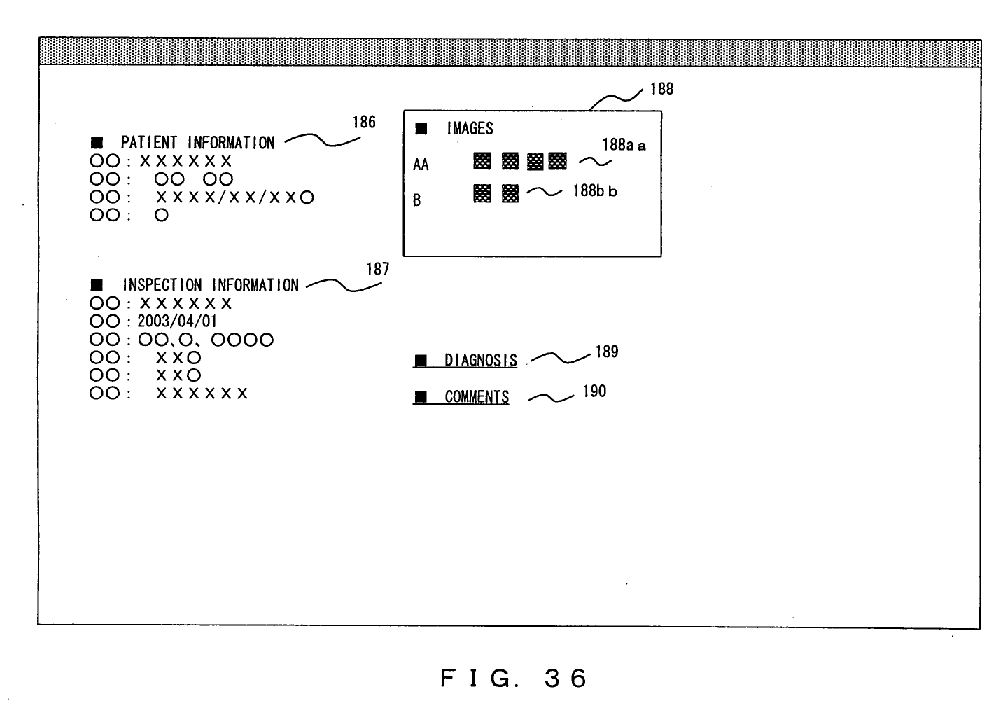

FIG. 36 shows an example of the made report. In FIG. 36, a report stored in the server 149 is displayed on the screen of an information processing device, such as the terminal 150 or the like as an example.

The report shown in FIG. 36 includes patient information 186 for indicating the personal information of a patient that took an endoscopic inspection (such as a patient ID, a name, the date of birth, sex, etc.), inspection information 187 for indicating the record of an endoscopic inspection to be reported, such as an inspection ID, the date of inspection, a body part to be inspected, the number of images, inspection time, inspection conditions, images 188 taken in during endoscopic inspection, diagnosis 189 and the comments 190 of a report maker. In the example, five esophagus images 188a and two stomach images 188b are selected and attached to the report. Each of the five esophagus images 188a and two stomach images 188b can be enlarged and displayed on the display screen by selecting it by a pointing device, and the contents of each of the diagnosis 189 and comments 190 can also be display by selection it.

In this system, when making such a report, endoscopic images to be attached to images 188 can be selected beforehand by marking images taken in at the time of endoscopic inspection. Therefore, labor needed to select images when making a report can be reduced.

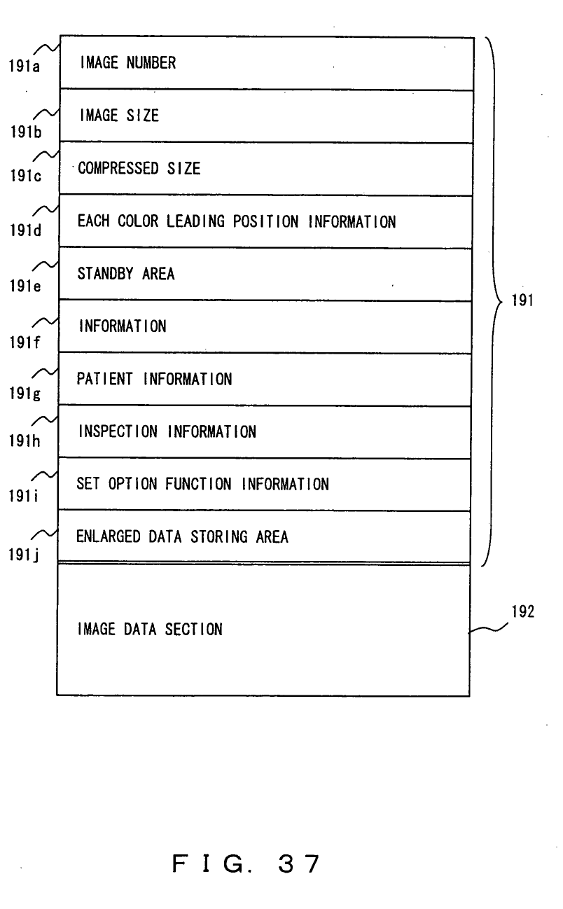

FIG. 37 shows an example of the structure of the image file for storing image data when transmitting endoscopic images registered in the server 149 from the image storage device 145.

In FIG. 37, the image file comprises a header section 191 and an image data section 192 for storing compressed image data. The header section 191 stores a variety of information about image data stored in the image data section 192. In the example, the header section contains an image number 191a for identifying each image, the size (image size) 191b of image data, compressed size 191c, each color leading position information 191d for indicating the leading position of each color of image data, a standby area 191e, information 191f, such as the name of an inspection device used in an endoscopic inspection, patient information 191g of an inspected patient, such as a patient ID, a patient name, age, sex and the like, inspection information 191h for indicating inspection conditions, such as the version of a storage device, an inspection date, an inspection number and the like, set option function information 191i for indicating the information about option settings, such as the lightness of a scope light source, enhancement and the like and an enlarged data storing area 191j. Flag information indicating whether an image is marked (existence/non-existence of a mark) is stored in the enlarged data storing area 191j.

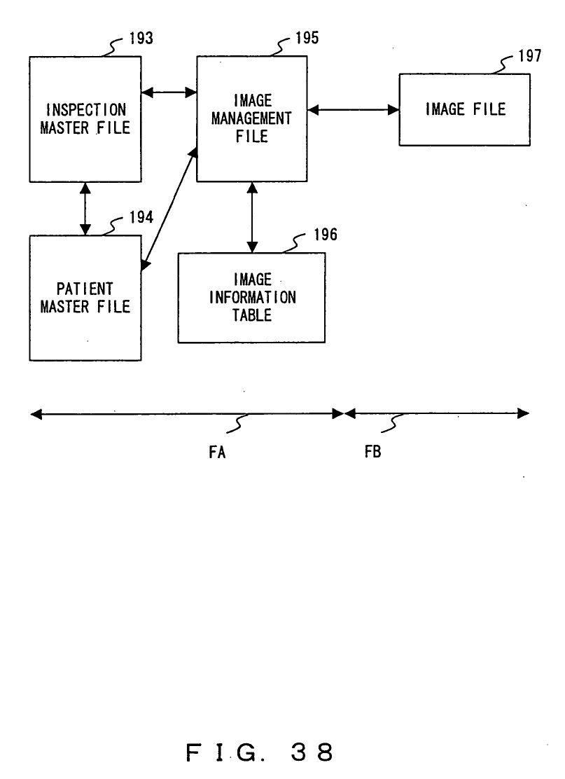

FIG. 38 shows an example of structure of the file in the server.

Endoscopic image data transmitted from the image storage device 145 to the server 149 is registered and stored in a database built on the server 149.

In FIG. 38, the endoscopic image data stored in the database is composed of an inspection master file 193, a patient master file 194, an image management file 195, an image information table 196 and an image file 197.

The image file 197 is composed of the image number 191a and the compressed image data in the image data section 192, which are shown in FIG. 37. Each of the inspection master file 193, patient master file 194, image management file 195 and image information table 196 is built based on a variety if information stored in the header section 191.

The inspection master file 193 is a table for managing information about an endoscopic inspection, such as an inspection date, an inspection time and the like. Inspection start/end times transmitted from the image storage device 145 are managed by this inspection master file 193. The patient master file 194 is a table for managing information about an inspected patient, such as a patient ID, a patient name and the like. The image management file 195 is a table for managing information about a taken endoscopic image, such as an inspection ID, a patient ID, an image number and the like (patient data checking image management table). The image information table 196 is a table for managing marking information, such as the image number of a marked image and the like. The image file 197 stores taken endoscopic image data and records compressed image data and its image number.

Of the information, the inspection master file 193 and patient master file 194, the inspection master file 193 and image management file 195, and the image management file 195 and the image information table 196 are linked by an inspection ID. The patient master file 194 and image management file 195 are linked by a patient ID.

In FIG. 38, the range covered by an arrow mark FA and the range covered by an arrow mark FB indicate a database and an image file, respectively.

When making a report, a report writer calls up the image management file 195 by an inspection ID via the inspection master file or by an patient ID via the patient master file 194, and displays an image selection screen containing the marking information shown in FIGS. 34 and 35 on the screen of the terminal 150, based on the image information table 196 and image file 197 which are linked with the image management file 195.

Then, the selected images are attached to a made report by its image number.

According to this configuration, the operator can attach information indicating that an arbitrary taken-in endoscopic image is selected to an image to be used for a report by marking the image.

As described above, according to this fifteenth preferred embodiment of the present invention, the operator can attach information indicating that an arbitrary taken-in endoscopic image is selected to an image to be used for a report by marking the image. That is, images to be used for a report can be selected at the time of endoscopic inspection. Therefore, there is no need to depend on memory later. Accordingly, images can be accurately selected. Furthermore, the issuance of a report can be simplified and a load to issue it can be reduced.

Furthermore, when making a report, only images selected beforehand can be retrieved, thereby helping the image selection.

Furthermore, the start and end times of an inspection can be accurately recorded.

Although the present invention has been described in detail above, the application of the present invention is not limited to the above-mentioned preferred embodiments. Its variations and modifications are also possible as long as they do not deviate from the subject matter of the present invention.

As described above, according to the present invention, if a variety of medical devices are complexly synthesized and used, advantageous effects can be obtained compared with the prior art. Furthermore, by sharing data possessed by each medical device, such data can be effectively utilized and a time needed to make a report can be shortened to reduce a workload. Furthermore, when making a report, labor needed to select images to be used for a report can be reduced to simplify the issuance of the report. Furthermore, a time needed for an endoscopic inspection can be accurately recorded.

What is claimed is:

1. A communication method of a storage device for recording medical information, comprising:
receiving information from a plurality of devices for providing at least one of setting information, status information, inspection image information and patient information; and
transmitting at least one of the setting information and status information or information obtained by processing the information, via a network, in addition to the inspection image information and patient information.

2. The communication method of the storage device according to claim 1, wherein
the received information or information obtained by processing the information is transmitted to a server device via the network, and is collectively managed by the server device.

3. The communication method of the storage device according to claim 1, wherein
the received inspection image information is recorded in at least one image recording format of a plurality of different image recording formats.

4. The communication method of the storage device according to claim 3, wherein
the recorded inspection image information is transmitted to a server via a network.

5. The communication method of the storage device according to claim 3, wherein
when recording the inspection image information, it is recorded in at least one image recording format selected and set from a plurality of the different image recording formats.

6. The communication method of the storage device according to claim 3, wherein
the plurality of different image recoding formats includes at least one of a unique image recording format, a DICOM-based image recording format and a JPEG format.

7. The communication method of the storage device according to claim 1, wherein
the received inspection image information and patient information are converted into inspection image information and patient information according to one communication protocol of a plurality of different communication protocols and are transmitted.

8. The communication method of the storage device according to claim 7, wherein
the transmitted inspection image information and patient information are transmitted to a network operated by each corresponding communication protocol.

9. The communication method of the storage device according to claim 7, wherein
when transmitting the inspection image information and patient information, they are converted into inspection image information and patient information according to one of a plurality of communication protocols, selected and set from the plurality of different communication protocols and are transmitted.

10. The communication method of the storage device according to claim 7, wherein
the plurality of different communication protocols includes at least one of a unique image recording format and a DICOM-based image recording format.

11. The communication method of the storage device according to claim 1, wherein
when starting the storage device, the received and stored history information that is provided by the plurality of devices is transmitted to a server device via a network.

12. The communication method of the storage device according to claim 1, wherein
image information about a thumbnail with a size selected and set from a plurality of different sizes is generated as image information about a thumbnail which aims to select images to be viewed.

13. The communication method of the storage device according to claim 1, wherein
image information about a thumbnail with a size corresponding to equipment information obtained from a device connected to the image storage device is generated as image information about a thumbnail which aims to select images to be viewed.

14. The communication method of the storage device according to claim 1, wherein
it is set which is used with priority, patient information inputted via an endoscope device included in the plurality of devices or patient information retrieved from a database.

15. The communication method of the storage device according to claim 1, wherein
a temporary patient ID using information peculiar to the storage device is attached to image information to be recorded.

16. The communication method of the storage device according to claim 15, wherein
the information peculiar to the image storage device is a serial number or MAC address of the image storage device.

17. The communication method of the storage device according to claim 15, wherein
the information peculiar to the image storage device is a serial number or MAC address of the image storage device with either or both of a release number and a record start date/time attached to it.

18. The communication method of the storage device according to claim 1, wherein
an inspection start time and an inspection end time are obtained based on an inspection start command and an inspection end command, respectively, transmitted from a selected and set device.

19. The communication method of the storage device according to claim 1, wherein
when transferring original image information, it is transferred for each dot of array of R, G and B or for each plane of R, G and B, according to selection and setting.

20. An image storage device system for recording medical information, comprising:
a plurality of devices for providing at least one of setting information, status information, inspection image information and patient information; and
an image storage device provided with a communication unit for transmitting/receiving information among the plurality of devices.

21. The image storage system according to claim 20, further comprising
a plurality of network systems whose corresponding image recording formats are different, wherein
the image storage device records the inspection image information received by the communication unit in at least one of a plurality of different image recording formats, and transmits the recorded inspection image information to a server device included in the corresponding network system.

22. The image storage system according to claim 21, wherein
the plurality of different image recoding formats includes at least one of a unique image recording format, a DICOM-based image recording format, and a JPEG format.

23. The image storage system according to claim 20, further comprising
a plurality of network systems whose corresponding communication protocols are different, wherein
the image storage device converts the inspection image information and patient information which are received by the communication unit into inspection image information and patient information, respectively, according to at least one of a plurality of different communication protocols and transmits them to a server device included in the corresponding network system.

24. The image storage system according to claim 23, wherein
the plurality of different communication protocols includes at least one of a unique image recording format and a DICOM-based image recording format.

25. The image storage system according to claim 20, further comprising
a server device for recording history information of the plurality of devices, wherein
the image storage device further comprises
a history information storage unit for recording history information of the plurality of devices, received by the communication unit,
and when starting the image storage device, transmits the history information of the plurality of devices, recorded in the history information storage unit, to the server device.

26. The image storage system according to claim 20, further comprising
a server device for recording image information about a thumbnail which aims to select images to be viewed;
an image replay device for replaying image information about a thumbnail recorded in the server device, wherein
the image storage device generates image information about a thumbnail with a size selected and set from a plurality of different sizes, and transmits it to the server device.

27. The image storage system according to claim 26, wherein
the image storage device generates image information about a thumbnail with a corresponding size as image information about the thumbnail, according to equipment information received from a device or the image replay device connected to the image storage device, and transmits it to the server device.

28. The image storage system according to claim 20, further comprising
an external storage device for recording patient information, wherein
the image storage device further comprises
first memory for recording patient information inputted via an endoscope device included in the plurality of devices;
second memory for recording patient information recorded in the external storage device; and
third memory for recording information indicating which is used with priority, the patient information recorded in the first memory or the patient information recorded in the second memory.

29. The image storage system according to claim 20, further comprising
an external storage device for recording image information, wherein
the image storage device further comprises
memory for recording information peculiar to the image storage device, and
a temporary patient ID using the information peculiar to the image storage device, recorded in the memory is attached to image information to be recorded in the external storage device.

30. The image storage system according to claim 29, wherein
the information peculiar to the image storage device is a serial number or MAC address of the image storage device.

31. The image storage system according to claim 29, wherein
the information peculiar to the image storage device is a serial number or MAC address of the image storage device with either or both of a release number and a record start date/time attached to it.

32. The image storage system according to claim 20, wherein
the image storage device further comprises
a setting unit for setting a device for receiving with priority an inspection start command and an inspection end command from the plurality of connected devices,
obtains an inspection start time and an inspection end time, based on an inspection start command and an inspection end command received from a device set by the setting unit.

33. The image storage system according to claim 20, wherein
the image storage device further comprises
original image memory for recording original image information;
a selector for switching between a first transfer route for transferring for each dot of array of R, G and B and a second transfer route for transferring for each plane of R, G and B, as a transfer route of the original image information recorded in the original image memory; and
a storage unit for recording original image information outputted from the transfer route switched by the selector.

34. A medical image storage device, comprising:
an endoscopic observation image storage unit for recording endoscopic observation image data obtained by an endoscope;
an endoscope insertion length storage unit for recording endoscope insertion length data indicating an insertion length of the endoscope in a body to be inspected; and
a data management unit for monitoring/managing endoscopic observation image data and endoscope insertion length data.

35. The medical image storage device according to claim 34, wherein
when specific observation image data is specified among a plurality of pieces of observation image data obtained by the endoscope, the data management unit specifies a corresponding body part in a sketch showing an outline of a body part to be inspected based on the endoscope insertion length data, links the corresponding body part in the sketch with the specific specified observation image data and displays them.

36. A medical image storage device, comprising:
endoscopic observation image recording means for recording endoscopic observation image data obtained by an endoscope;
endoscope insertion length recording means for recording endoscope insertion length data indicating an insertion length of an endoscope in a body to be inspected; and
data management means for monitoring/managing endoscopic observation image data and endoscope insertion length data.

37. A medical image storage device for recording endoscopic images shot by an endoscope comprising:
an image sensing unit for taking in an endoscopic image shot by the endoscope as image data, according to an instruction of an operator; and
a marking unit for attaching information indicating that the endoscopic image taken in by the image sensing unit is selected, to the endoscopic image, according to an instruction of the operator.

38. The medical image storage device according to claim 37, further comprising:
a mark generation unit for generating a mark image; and
a synthesis unit for synthesizing the endoscopic image from the endoscope and the mark image and outputting it to a monitor.

39. The medical image storage device according to claim 37, further comprising
an inspection time storage unit for recording a start time and an end time of an endoscopic inspection, according to an instruction of the operator.

40. The medical image storage device according to claim 37 which is connected to a server device via a network, further comprising
a transmitting unit for transmitting image data of the endoscopic image taken in by the image sensing unit to the server device via the network.

41. The medical image storage device according to claim 37, wherein
an image attached to an electronic carte or an inspection report is determined based on information attached by the marking unit.

42. A medical image storage device for recording endoscopic images shot by an endoscope comprising:
image sensing means for taking in an endoscopic image shot by the endoscope as image data, according to an instruction of an operator; and
marking means for attaching information indicating that the endoscopic image taken in by the image sensing means is selected, to the endoscopic image, according to an instruction of the operator.

43. A method for taking in an endoscopic image of a medical image storage device for recording an endoscopic image shot by an endoscope, comprising:
taking in the endoscopic image shot by the endoscope as image data, according to an instruction of an operator; and
attaching information indicating that the taken endoscope image is selected, to the taken endoscopic image, according to an instruction of the operator.

44. A computer-readable storage medium, on which a program for enabling a computer of an information processing device for recording an endoscopic image shot by an endoscope to perform a function, the function comprising:
taking in the endoscopic image shot by the endoscope as image data, according to an instruction of an operator; and
attaching information indicating that the taken endoscope image is selected, to the taken endoscopic image, according to an instruction of the operator.
